SEQUENCE LISTING (23 sequences)

SEQ ID NO: 1 - DNA, 19 bp, synthetic construct
  source 1..19: mol_type = other DNA; note = shRNA to mouse SLC30A1
ttgatatcgatttgcttag

SEQ ID NO: 2 - DNA, 19 bp, synthetic construct
  source 1..19: mol_type = other DNA; note = shRNA to mouse SLC30A1
ttacttgtacatccactgg

SEQ ID NO: 3 - protein, 507 aa, Homo sapiens
MGCWGRNRGRLLCMLALTFMFMVLEVVVSRVTSSLAMLSDSFHMLSDVLALVVALVAERFARRTHATQKNTFGWIRAEVMGALVNAIFLTGLCFAILLEAIERFIEPHEMQQPLVVLGVGVAGLLVNVLGLCLFHHHSGFSQDSGHGHSHGGHGHGHGLPKGPRVKSTRPGSSDINVAPGEQGPDQEETNTLVANTSNSNGLKLDPADPENPRSGDTVEVQVNGNLVREPDHMELEEDRAGQLNMRGVFLHVLGDALGSVIVVVNALVFYFSWKGCSEGDFCVNPCFPDPCKAFVEIINSTHASVYEAGPCWVLYLDPTLCVVMVCILLYTTYPLLKESALILLQTVPKQIDIRNLIKELRNVEGVEEVHELHVWQLAGSRIIATAHIKCEDPTSYMEVAKTIKDVFHNHGIHATTIQPEFASVGSKSSVVPCELACRTQCALKQCCGTLPQAPSGKDAEKTPAVSISCLELSNNLEKKPRRTKAENIPAVVIEIKNMPNKQPESSL

SEQ ID NO: 4 - DNA, 19 bp, synthetic construct
  misc_feature 1..19: note = therapeutic RNA
  source 1..19: mol_type = other DNA
tgataattgtcactgacag

SEQ ID NO: 5 - protein, 460 aa, Homo sapiens
MAPGRAVAGLLLLAAAGLGGVAEGPGLAFSEDVLSVFGANLSLSAAQLQHLLEQMGAASRVGVPEPGQLHFNQCLTAEEIFSLHGFSNATQITSSKFSVICPAVLQQLNFHPCEDRPKHKTRPSHSEVWGYGFLSVTIINLASLLGLILTPLIKKSYFPKILTFFVGLAIGTLFSNAIFQLIPEAFGFDPKVDSYVEKAVAVFGGFYLLFFFERMLKMLLKTYGQNGHTHFGNDNFGPQEKTHQPKALPAINGVTCYANPAVTEANGHIHFDNVSVVSLQDGKKEPSSCTCLKGPKLSEIGTIAWMITLCDALHNFIDGLAIGASCTLSLLQGLSTSIAILCEEFPHELGDFVILLNAGMSTRQALLFNFLSACSCYVGLAFGILVGNNFAPNIIFALAGGMFLYISLADMFPEMNDMLREKVTGRKTDFTFFMIQNAGMLTGFTAILLITLYAGEIELE

SEQ ID NO: 6 - DNA, 20 bp, synthetic construct
  source 1..20: mol_type = other DNA; note = primer
caacaccagcaattccaacg

SEQ ID NO: 7 - DNA, 23 bp, synthetic construct
  source 1..23: mol_type = other DNA; note = primer
tgtcagactcctggatgagattc

SEQ ID NO: 8 - DNA, 19 bp, synthetic construct
  source 1..19: mol_type = other DNA; note = primer
gccagctgcacttcaacca

SEQ ID NO: 9 - DNA, 24 bp, synthetic construct
  source 1..24: mol_type = other DNA; note = primer
agagaagttcgagctggttatctg

SEQ ID NO: 10 - DNA, 23 bp, synthetic construct
  source 1..23: mol_type = other DNA; note = primer
tttaacgatggcctgaatcactt

SEQ ID NO: 11 - DNA, 23 bp, synthetic construct
  source 1..23: mol_type = other DNA; note = primer
cagcacaatcacgatcatattgc

SEQ ID NO: 12 - DNA, 21 bp, synthetic construct
  source 1..21: mol_type = other DNA; note = primer
cgccgctagaggtgaaattct

SEQ ID NO: 13 - DNA, 21 bp, Synthetic construct
  source 1..21: mol_type = other DNA; note = primer
cgaacctccgactttcgttct

SEQ ID NO: 14 - DNA, 21 bp, synthetic construct
  source 1..21: mol_type = other DNA; note = primer
tcctgaggagaagtctgccgt

SEQ ID NO: 15 - DNA, 21 bp, synthetic construct
  source 1..21: mol_type = other DNA; note = primer
ggagtggacagatccccaaag

SEQ ID NO: 16 - DNA, 19 bp, synthetic construct
  source 1..19: mol_type = other DNA; note = primer
gggtggacccggtcaactt

SEQ ID NO: 17 - DNA, 18 bp, synthetic construct
  source 1..18: mol_type = other DNA; note = primer
gaggtgggcggccagggt

SEQ ID NO: 18 - DNA, 21 bp, synthetic construct
  source 1..21: mol_type = other DNA; note = primer
ccaataccagcaactccaacg

SEQ ID NO: 19 - DNA, 25 bp, synthetic construct
  source 1..25: mol_type = other DNA; note = primer
tacttccactgtatcaccacttctg

SEQ ID NO: 20 - DNA, 19 bp, synthetic construct
  source 1..19: mol_type = other DNA; note = primer
gccagctgcacttcaacca

SEQ ID NO: 21 - DNA, 24 bp, synthetic construct
  source 1..24: mol_type = other DNA; note = primer
gtaagactgctggacagatgacag

SEQ ID NO: 22 - DNA, 18 bp, synthetic construct
  source 1..18: mol_type = other DNA; note = primer
ggccaaccgcgagaagat

SEQ ID NO: 23 - DNA, 21 bp, synthetic construct
  source 1..21: mol_type = other DNA; note = primer
ccagaggcgtacagggatagc

(19) United States
(12) Patent Application Publication
(10) Pub. No.: US 2023/0295632 A1
(43) Pub. Date: Sep. 21, 2023

(54) METHODS OF CONTROLLING RED BLOOD CELL PRODUCTION

(71) Applicant: Wisconsin Alumni Research Foundation, Madison, WI (US)

(72) Inventors: Emery H. Bresnick, Middleton, WI (US); Noboyuki Tanimura, Kita-Ku (JP)

(21) Appl. No.: 18/304,694

(22) Filed: Apr. 21, 2023

Related U.S. Application Data
(63) Continuation of application No. 16/292,922, filed on Mar. 5, 2019.
(60) Provisional application No. 62/638,563, filed on Mar. 5, 2018.

(51) Int. Cl. C12N 15/113; C12N 5/078; A61K 33/30; A61K 45/06; A61K 31/7088; A61K 35/18; A61P 7/06

(52) U.S. Cl. CPC C12N 15/113; C12N 5/0641; A61K 33/30; A61K 45/06; A61K 31/7088; A61K 35/18; A61P 7/06; C12N 2310/14; C12N 2506/11; C12N 2501/999; C12N 2501/90; C12N 2310/122; C12N 2310/531; C12N 2506/03

(57) ABSTRACT

A method of controlling red blood cell production includes contacting red blood cell precursors with a composition including a zinc chelator, a composition including an inhibitor of a zinc importer protein, or a combination thereof; wherein the contacting inhibits survival of the red blood cell precursors, inhibits terminal differentiation of the red blood cell precursors to mature red blood cells, or a combination thereof.

### CROSS-REFERENCE TO RELATED APPLICATIONS

This application is a continuation of U.S. application Ser. No. 16/292,922, filed Mar. 5, 2019, which claims priority to U.S. Provisional Application 62/638,563 filed on Mar. 5, 2018, which are incorporated herein by reference in their entirety.

### STATEMENT REGARDING FEDERALLY SPONSORED RESEARCH & DEVELOPMENT

This invention was made with government support under DK050107 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Apr. 19, 2023 is named WIS0048USC and is 26,036 bytes in size.

### FIELD OF THE DISCLOSURE

The present disclosure is related to methods of controlling red blood cell production as well as methods of treating subjects suffering from a disease associated with defective erythropoiesis or subjects in need of red blood cell transplants.

### BACKGROUND

Erythropoiesis is the process by which red blood cells (erythrocytes) develop and differentiate from hematopoietic stem cells in the bone marrow. This process involves a complex interplay of polypeptide growth factors (cytokines and hormones) acting via membrane-bound receptors on the target cells. Cytokine action results in cellular proliferation and differentiation, with response to a particular cytokine often being stage-specific. The two most prominent cytokines that regulate erythropoiesis are erythropoietin (Epo) and stem cell factor (SCF; also referred to as mast cell growth factor [MGF], Steel factor [SLF], or Kit ligand [KL]). Erythropoietin (Epo) is a protein hormone that acts in concert with other growth factors, such as SCF, to stimulate the proliferation and maturation of responsive bone marrow erythroid precursor cells.

Anemias are a common disorder of erythropoiesis and are the result of an insufficient number of erythrocytes. Anemia results in decreased oxygen transport capacity that can lead to impaired physical activity, organ failure, or death. Chronic progressive anemias result from renal disease, AIDS, iron transport deficiencies, chronic inflammation, and as a side effect of cytoreductive cancer therapies. Other chronic anemias result from congenital disorders of erythropoiesis itself or because factors needed to stimulate erythropoiesis are missing due to a genetic disorder. Acute anemia results from surgery or trauma resulting in a rapid or large blood loss. Treatment of anemia is required once hematocrits (the % of blood mass made up of erythrocytes) drop below 30%.

What is needed is a detailed biochemical and molecular characterization of erythropoiesis to determine how cellular processes are regulated and de-regulated in disease, which will lead to new treatments for diseases such as anemias.

### BRIEF SUMMARY

In an aspect, a method of controlling red blood cell production comprises contacting red blood cell precursors with a composition comprising zinc, a composition comprising an inhibitor of a zinc exporter protein, or a combination thereof; wherein the contacting promotes survival of the red blood cell precursors, promotes terminal differentiation of the red blood cell precursors to mature red blood cells, or a combination thereof;

or contacting red blood cell precursors with a composition comprising a zinc chelator, a composition comprising an inhibitor of a zinc importer protein, or a combination thereof; wherein the contacting inhibits survival of the red blood cell precursors, inhibits terminal differentiation of the red blood cell precursors to mature red blood cells, or a combination thereof.

In another aspect, a method of treating a patient suffering from a disease associated with defective erythropoiesis comprises
administering an effective amount of a composition comprising zinc, a composition comprising an inhibitor of a zinc exporter protein, or a combination thereof;
wherein the administering promotes survival of red blood cell precursors, promotes terminal differentiation of red blood cell precursors to mature red blood cells, or a combination thereof;
or
administering an effective amount of a composition comprising a zinc chelator, a composition comprising an inhibitor of a zinc importer protein, or a combination thereof;
wherein the administering inhibits survival of red blood cell precursors, inhibits terminal differentiation of red blood cell precursors to mature red blood cells, or a combination thereof.

In yet another aspect, a method of treating a human subject in need of a red blood cell transplant comprises
ex vivo culturing of hematopoietic precursor cells or pluripotent stem cells in the presence of a composition comprising zinc, a composition comprising an inhibitor of a zinc exporter protein, e.g., SLC30A1, or a combination thereof; wherein contacting either promotes survival of hematopoietic precursor cells or precursors from pluripotent stem cells, or promotes terminal differentiation of the hematopoietic precursor cells or precursors from pluripotent stem cells to mature, differentiated red blood cells;
isolating mature, differentiated red blood cells from the ex vivo culture; and
transplanting the mature, differentiated red blood cells into the human subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-F show GATA/heme multi-omics in a GATA-1 genetic complementation system with normal and sub-physiological heme. FIG. 1A shows a genetic complementation assay. β-estradiol activation of ER-GATA-1 in GATA-1-null G1E-ER-GATA-1 cells induces Alas2 expression, which increases heme biosynthesis in WT cells. Alas2 intron ⅛ mutations in mutant cells (double mutant) severely reduced Alas2 expression and heme levels. FIG. 1B shows volcano plots of quantitative proteomics data depicting GATA-1-regulated proteins identified by comparing uninduced and β-estradiol-treated WT G1E-ER-GATA-1 cell proteomes and Alas2-enhancer-regulated proteins identified by comparing 13-estradiol-treated WT and double mutant cell proteomes (n=3 biological replicates). FIG. 1C shows Venn diagrams depicting overlap between GATA-1-activated and -repressed proteins and mRNA. FIG. 1D shows Venn diagrams depicting overlap between Alas2-enhancer-activated and -repressed proteins and mRNA. FIG. 1E shows the peptide intensities of representative proteins (n=3 biological replicates, mean+/−SE). FIG. 1F shows that 11 genes are activated by both GATA-1 and Alas2 enhancer at the protein and mRNA levels. The 11 proteins activated by GATA-1/Alas2 enhancer are shown in volcano plots. See also FIG. 2.

FIG. 2 shows a principal component analysis of non-treated and β-estradiol-treated WT and double mutant G1E-ER-GATA-1 cell proteomes (n=3 biological replicates).

FIGS. 3A-C show that a GATA-1/heme circuit confers expression of the predominant zinc exporter and importer in erythroblasts. FIG. 3A shows an expression heatmap for zinc exporters (Slc30 family) and importers (Slc39 family) identified by comparing control and β-estradiol-treated WT G1E-ER-GATA-1 cell transcriptomes. FIG. 3B shows a real-time RT-PCR analysis of mRNA levels of Slc30a1 and Slc39a8 in two WT and double mutant G1E-ER-GATA-1 clones (n=4 biological replicates from two independent experiments, mean+/−SE). All values of β-estradiol-treated WT1 and 3 were compared to all values of β-estradiol-treated double mutant 1 and 2 clones. P values were calculated by unpaired two-tailed t-test. *** P<0.001, NS: not significant. FIG. 3C shows peptide intensities of zinc transporter proteins (n=3 biological replicates from one experiment, mean+/−SE). P values were calculated by one-way ANOVA, followed by Tukey's test. *P<0.05, ** P<0.01, *** P<0.001, NS: not significant.

FIGS. 4A-F show a zinc transporter switch during primary mouse and human erythroid differentiation. FIG. 4A shows representative flow cytometric plots of CD71 and Ter119 from lineage-negative cells isolated from E14.5 fetal livers and cultured for 3 days. The percentage of each population is shown (n=6 biological replicates from two independent experiments, mean+/−SE). FIG. 4B shows the relative expression of Hbb-b1, Slc30a1, and Slc39a8 in each population (n=6 biological replicates from two independent experiments, mean+/−SE). FIG. 4C shows a schematic diagram for differentiation of human mononuclear cells from G-CSF-mobilized peripheral blood. Representative flow cytometric plots of CD71 and CD235a, and the percentage of each population at each stage are shown (n=4 biological replicates from two independent experiments, mean+/−SE). Concentration of heme per cell treated with or without a heme biosynthesis inhibitor succinylacetone (SA) at day 20 was quantitated (n=2 biological replicates from one experiment, mean+/−SD). FIG. 4D shows representative photomicrographs of Wright-Giemsa staining of cells treated with or without SA. Scale bar, 20 μm. FIG. 4E shows the relative expression of HBB, HBA1, SLC30A1, and SLC39A8 during differentiation (n=4 biological replicates from two independent experiments, mean+/−SE). FIG. 4F shows the relative expression of HBB, HBA1, SLC30A1, SLC39A8, and ACTB with SA treatment in comparison with no treatment at day 19 and 20 (n=4 biological replicates from two independent experiments, mean+/−SE). P values were calculated by unpaired two-tailed t-test. ** P<0.01, *** P<0.001, NS: not significant.

FIG. 5 shows the expression levels of all Slc30 (zinc exporter) and Slc39 (zinc importer) family members in mouse and human proerythroblasts and orthochromatic erythroblasts.

FIGS. 6A-F show that the zinc transporter switch dynamically controls intracellular zinc during erythroid maturation. FIG. 6A shows representative flow cytometric plots of CD71, Ter119 and FSC-A from uncultured E14.5 fetal liver cells. FIG. 6B shows representative flow cytometric plots of FluoZin™3 and FSC-A and histograms of FluoZin™3 in each population. Quantitation of FluoZin™-3 MFI (Median Fluorescence Intensity) in each population (n=4 biological replicates from two independent experiments, mean+/−SE). P values were calculated by repeated-measures one-way ANOVA, followed by Tukey's test. *P<0.05. FIG. 6C shows representative flow cytometric plots of CD71, Ter119 and FSC-A from uncultured bone marrow cells. FIG. 6D shows representative flow cytometric plots of FluoZin™-3 and FSC-A and histograms of FluoZin-3™ in each population. FluoZin™3 MFI in each population was quantitated (n=4 mice from two independent experiments, mean+/−SE). P values were calculated by repeated-measures one-way ANOVA, followed by Tukey's test. **P<0.01. FIG. 6E shows representative flow cytometric plots of CD71, CD235a, and FSC-A from differentiated human mononuclear cells from G-CSF-mobilized peripheral blood at day 13, and FIG. 6F shows day 16. Representative flow cytometric plots of FluoZin™-3 and FSC-A and histograms of FluoZin™-3 in each population are shown. FluoZin™3 MFI in each population was quantified (n=4 biological replicates from two independent experiments, mean+/−SE). P values were calculated by repeated-measures one-way ANOVA, followed by Tukey's test. **P<0.01, *** P<0.001.

FIG. 7 shows increased intracellular zinc during initial phases of primary human erythroblast maturation. Representative flow cytometric plots of CD71 and CD235a from differentiated human mononuclear cells from G-CSF-mobilized peripheral blood at day 9 are shown. Representative flow cytometric plots of FluoZin™-3 and FSC-A and histograms of FluoZin™-3 in each population are shown. FluoZin™3 MFI in each population was quantified (n=4 biological replicates from two independent experiments, mean+/−SE). P values were calculated by paired two-tailed t-test. *** P<0.001

FIGS. 8A-F show a zinc-dependent cellular survival mechanism. FIG. 8A shows a schematic of the experiments using the zinc chelator TPEN and ZnCl₂. FIG. 8B shows representative flow cytometric plots of FSC-A, SSC-A, Live/dead dye, CD71, and Ter119 at day 3. FIG. 8C shows quantitation of FluoZin™3 MFI in each population of untreated and TPEN- or TPEN/ZnCl₂-treated cells. P values were calculated by one-way ANOVA followed by Tukey's test (n=4 biological replicates from two independent experiments, mean+/−SE). *P<0.05, *** P<0.001. FIG. 8D shows quantitation of total live populations of untreated and TPEN- or TPEN/ZnCl₂-treated cells. P values were calculated by one-way ANOVA followed by Tukey's test (n=4 biological replicates from two independent experiments, mean+/−SE). *** P<0.001. FIG. 8E shows the percentage of R1, R2, CD71^{high}FSC-A^{high} and CD71^{high}FSC-A^{high} populations in untreated and TPEN- or TPEN/ZnCl₂-treated cells. P values were calculated by one-way ANOVA followed by Tukey's test (n=4 biological replicates from two independent experiments, mean+/−SE). ** P<0.01, *** P<0.001, NS: not significant. FIG. 8F shows a Spearman rank correlation between intracellular zinc levels and sensitivity to TPEN-induced cell death represented as percentage decrease of each population caused by TPEN treatment. Each symbol represents one biological replicate (n=4 biological replicates from two independent experiments). r's: Spearman's rank correlation coefficient.

FIGS. 9A-E show that corrupting zinc regulation promotes erythroid differentiation. FIG. 9A shows Slc30a1 and Slc39a8 knockdown in lineage-negative hematopoietic precursors (E14.5) expanded for 2 days or expanded for 2 days and then differentiated for 3 days (n=4 biological replicates from two independent experiments, mean+/−SE). P values were calculated by one-way ANOVA followed by Dunnett's test (n=4 biological replicates from two independent experiments, mean+/−SE). *** P<0.001. FIG. 9B shows representative flow cytometric plots of FluoZin™3 and FSC-A in Slc30a1- or Slc39a8-knockdown lineage-negative hematopoietic precursors expanded for 2 days (day 2) or expanded for 2 days and then differentiated for 3 days (day 5). The percentage of high- and low-zinc populations at day 2 and 5 were shown (n=4 biological replicates from two independent experiments, mean+/−SE). P values were calculated by unpaired two-tailed t-test. ** P<0.01, *** P<0.001. FIG. 9C shows representative flow cytometric plots of CD71, Ter119, and FSC-A at day 2 and 5. FIG. 9D shows quantitation of FluoZin™3 MFI in each population of control and Slc30a1- or Slc39a8-knockdown cells at day 2 (top) and day 5 (bottom). P values were calculated by one-way ANOVA followed by Dunnett's test (n=4 biological replicates from two independent experiments, mean+/−SE). *P<0.05, ** P<0.01, *** P<0.001. FIG. 9E shows the percentage of CD71^{high}FSC-A^{low} and CD71^{low}FSC-A^{low} populations in control and Slc30a1- or Slc39a8-knockdown cells. P values were calculated by one-way ANOVA followed by Dunnett's test (n=4 biological replicates from two independent experiments, mean+/−SE). ** P<0.01, *** P<0.001.

FIG. 10 shows increased and decreased intracellular zinc during cultured murine erythroblast maturation. Representative histograms of FluoZin-3 in each population are shown. FluoZin™-3 MFI in each population was quantified (n=4 biological replicates from two independent experiments, mean+/−SE). P values were calculated by repeated-measures one-way ANOVA followed by Tukey's test. *P<0.05, ***P<0.01; ***P<0.001.

FIGS. 11A-D show intracellular zinc as a determinant of terminal differentiation. FIG. 11A shows representative flow cytometric plots of CD71, Ter119, and thiazole orange (TO) in Slc30a1- or Slc39a8-knockdown lineage-negative hematopoietic precursors expanded for 2 days and then differentiated for 3 days. FIG. 11B shows the percentage of CD71^{high}TO^{high}, CD71^{low}TO^{high}, and CD71^{low}TO^{low} populations in control and Slc30a1- or Slc39a8-knockdown cells. P values were calculated by one-way ANOVA, followed by Dunnett's test (n=4 biological replicates from two independent experiments, mean+/−SE). ** P<0.01, *** P<0.001. FIG. 11C shows the ratios of enucleated cell numbers divided by nucleated cell numbers in control and Slc30a1-knockdown cells (n=2 biological replicates from one experiment, mean+/−SD). FIG. 11D shows representative photomicrographs of Wright-Giemsa staining of control and Slc30a1-knockdown cells. Scale bar, 10 μm.

FIG. 12 shows GATA-1/heme-dependent zinc transporter switch controls intracellular zinc as a mechanism governing cellular differentiation. Changes in heme, Slc30a1, Slc39a8 and intracellular zinc levels during erythroid differentiation are depicted as heat maps. The circuits at the bottom illustrate the heme-independent, GATA-1-mediated induction of the zinc importer Slc39a8 early in erythroid differentiation to increase intracellular zinc. Reducing intracellular zinc via chelation severely compromised cell survival. As GATA-1 activates Alas2 transcription, which greatly increases heme, heme amplifies GATA-1 activity to induce the zinc exporter Slc30a1. Subsequently, Slc30a1 expression persists and Slc39a8 expression declines, which we term a zinc transporter switch. This switch is responsible for a steep decline in intracellular zinc during terminal differentiation. Corrupting the switch by downregulating Slc30a1 increases intracellular zinc and promotes terminal differentiation. By decommissioning zinc-dependent pro-differentiation processes, this decline may ensure high fidelity of the final cellular remodeling steps required to form the reticulocyte, while ensuring that precursors are not depleted upon differentiation, thus avoiding anemia. These results support a model in which the transcription factor GATA-1 and the cofactor heme regulate levels of a trace metal to differentially control fundamental activities of erythrocyte precursors—to survive and to differentiate.

The above-described and other features will be appreciated and understood by those skilled in the art from the following detailed description, drawings, and appended claims.

### DETAILED DESCRIPTION

To achieve a global perspective on how metal-containing cofactors intermesh with transcriptional regulatory machinery and to unveil new dimensions into how cellular processes are regulated and de-regulated in disease, the inventors deployed a GATA/heme multi-omics strategy. The unique multi-omics resource that emerged established a paradigm in which intracellular zinc confers cellular survival, and a GATA/heme circuit instigates a zinc transporter switch that regulates cellular differentiation.

Specifically, the inventors developed a GATA/heme multi-omics resource that enabled the discovery of a zinc transporter switch and a trace metal-dependent cellular differentiation mechanism. Whereas trace metals are critical nutritional constituents, many questions remain regarding their requirements and mechanisms in fundamental aspects of cell regulation. Evidence for an evolutionarily conserved GATA/heme-dependent mechanism that dynamically regulates intracellular zinc during erythrocyte differentiation is provided (FIG. 12). GATA-1 activates expression of the predominant zinc importer and exporter of the erythroblast. As differentiation proceeds and heme accumulates, heme amplifies GATA-1-dependent exporter induction. Sustained exporter expression with importer loss constitutes a zinc transporter switch that reduces intracellular zinc during terminal differentiation. Corrupting this mechanism by zinc-deprivation compromises erythroblast survival, and exporter downregulation elevates intracellular zinc, mimicking the levels of early differentiation, and promotes terminal differentiation. By regulating erythroblast survival and differentiation, the GATA/heme/zinc switch controls erythrocyte differentiation.

The essential trace metal zinc has important structural, catalytic and signaling functions. The results provided herein raise the question of how the intracellular zinc binding protein ensemble senses intracellular zinc changes, such as those driven by GATA/heme that control differentiation. The key transcriptional regulators of erythrocyte differentiation, GATA-1 and KLF1, contain zinc fingers that mediate sequence-specific DNA binding and/or protein-protein interactions with coregulators. Zinc increases activity of the zinc-finger transcription factor Metal Transcription Factor 1 (MTF-1), which activates transcription of zinc-regulatory genes, including metallothioneins, intracellular zinc-chelating proteins. The octameric ALAD heme biosynthesis enzyme contains eight zinc atoms required for enzymatic activity. While iron insertion into protoporphyrin IX forms heme physiologically, in iron deficiency, zinc replaces iron to yield zinc protoporphyrin IX. This zinc-containing heme analogue inhibits heme oxygenase, an enzyme that mediates heme catabolism and confers cytoprotection.

Concerted enzymatic reactions in mitochondria synthesize the iron-protoporphyrin molecule heme that controls essential cellular processes. Mutations of ALAS2, encoding the rate-limiting enzyme of heme synthesis aminolevulinic acid synthase II, disrupt heme synthesis and elicit deleterious phenotypes including anemia. Beyond the canonical heme functions as an iron-containing enzyme cofactor and oxygen-binding hemoglobin component, heme also controls gene expression. However, the physiological and pathological mechanisms underlying heme-regulated gene expression are incompletely understood.

Iron and heme regulate gene expression transcriptionally and post-transcriptionally. Iron activates iron response element binding proteins (IRPs) that form complexes on iron response elements (IREs) of target mRNAs. This system represses ALAS-2 protein translation. As an imbalance between hemoglobin polypeptides and heme is pathogenic, coordinating globin gene expression and heme synthesis is critical. This coordination is accomplished, in part, via heme-mediated activation of an EIF2a kinase that suppresses globin translation. Heme binds the repressor Bachl, inducing its degradation by the proteasome. In a low heme environment, Bachl accumulates and occupies cis-elements at target genes, including Hbb-b1 encoding β-globin, to repress transcription. Besides pathologies caused by insufficient heme, ablation of a heme exporter (FLVCR1) increases intracellular heme and impairs erythrocyte development.

In GATA-1-null erythroid precursor cells (G1E-ER-GATA-1), we used CRISPR/Cas9 to delete Alas2 intron 1 and 8 GATA motifs that bind GATA-1 (double mutants), the master regulator of erythrocyte differentiation. These cells stably express a β-estradiol-activated allele encoding the estrogen receptor hormone binding domain fused to GATA-1. In this genetic complementation system, the GATA motif mutations abrogated GATA-1-mediated activation of Alas2 transcription, decreased heme approximately 30 fold and attenuated activation of a GATA-1 target gene cohort. Bachl accumulates in low-heme conditions and opposes GATA-1-mediated activation of a gene cohort including β-globin. Heme amplification of additional GATA-1-activated genes was Bachl-independent. Through Bachl-dependent and -independent mechanisms, heme establishes/maintains the erythroblast transcriptome and controls differentiation.

The unique multi-omics resource that the inventors employed herein established a paradigm in which intracellular zinc confers cellular survival, and a GATA/heme circuit instigates a zinc transporter switch that regulates cellular differentiation.

Since erythroid differentiation involves a progressive increase, and terminal differentiation involves a sharp decline in intracellular zinc, it is of considerable interest to consider how variable zinc levels in differentiating systems impact members of the ensemble of zinc binding proteins (zinc sensors) and, presumably, unknown effectors. It is attractive to propose that the zinc accumulation early in differentiation commissions specific components of the ensemble to confer survival, while the zinc transporter switch decommissions factors that restrict differentiation. This decommissioning may function to balance zinc-stimulated processes early in differentiation with subsequent zinc-inhibited processes required for terminal differentiation. Accordingly, the dynamic regulation of intracellular zinc by GATA/heme is proposed to be a determinant of the precursor/progeny balance that ensures the generation of billions of erythrocytes daily without precursor depletion or inadequate progeny genesis, both of which would yield anemia.

Zinc deficiency in humans can be associated with anemia, is an independent risk factor for anemia in New Zealand children and plasma zinc correlates with hemoglobin levels in pregnant women from southern Ethiopia. Although zinc replacement has been deployed with apparent benefits as an anemia treatment in specific contexts, complexities arise with interpreting such studies, as zinc may exert a panoply of erythroid non-autonomous and cell-autonomous activities. Zinc deficiency in mice reduces erythroid and lymphoid cells, while increasing myeloid cells. Acute blood loss, which instigates protective mechanisms, increases bone marrow zinc levels. In aggregate, the zinc-dependent biochemical processes and correlative studies in humans suggest the precise control of zinc is important for erythrocyte differentiation. However, whether the links between zinc deficiency and anemia, and potential efficacy of zinc replacement reflect direct zinc actions in erythroid cells or non-erythroid-autonomous activities is unclear.

It is instructive to consider if altering the zinc exporter/importer balance impacts differentiation in diverse systems, including cancer cells with differentiation blockades that underlie malignant phenotypes. Furthermore, modulation of the zinc-dependent differentiation mechanism may surmount ineffective erythropoiesis, for example in the context of resistance to erythropoiesis-stimulating agents that can occur in MDS and other pathologies. Leveraging the mechanism to regulate intracellular zinc may have utility to promote terminal erythroid differentiation of pluripotent cells to achieve cell engineering goals for regenerative medicine.

In an aspect, a method of controlling red blood cell production comprises contacting red blood cell precursors with a composition comprising zinc, a composition comprising an inhibitor of a zinc exporter protein, or a combination thereof; wherein the contacting promotes survival of the red blood cell precursors, promotes terminal differentiation of the red blood cell precursors to mature red blood cells, or a combination thereof;
or
contacting red blood cell precursors with a composition comprising a zinc chelator, a composition comprising an inhibitor of a zinc importer protein, or a combination thereof; wherein the contacting inhibits survival of the red blood cell precursors, inhibits terminal differentiation of the red blood cell precursors to mature red blood cells, or a combination thereof.

Red blood cell precursors, known as erythroblasts, pass through several stages of differentiation as they develop to mature red blood cells. Red blood cell precursors have nuclei, while mature red blood cells do not. Thus, mature red blood cells cannot manufacture proteins such oxygen-carrying hemoglobin.

Thus, in an embodiment, red blood cell precursors are contacted with a composition comprising zinc, wherein contacting promotes survival of the red blood cell precursors, promotes terminal differentiation of the red blood cell precursors to mature red blood cells, or a combination thereof. Exemplary concentrations of zinc are 1 to 100 μM. The zinc can be in the form of a salt or compound, such as zinc nitrate, zinc sulfate, zinc chlorate, zinc phosphate, zinc acetate, zinc chloride, zinc sulfate, and the like.

In an embodiment, the red blood cell precursors are contacted with a composition comprising an inhibitor of a zinc exporter protein, e.g., SLC30A1; wherein contacting promotes survival of the red blood cell precursors, promotes terminal differentiation of the red blood cell precursors to mature red blood cells, or a combination thereof.

Exemplary inhibitors of the zinc exporter protein are therapeutic RNAs, e.g., the shRNAs of SEQ ID NOs: 1 and 2.

SEQ ID NO: 1
TTGATATCGATTTGCTTAG (Mouse)
SEQ ID NO: 2
TTACTTGTACATCCACTGG (Mouse)

In an aspect, the inhibitory RNA is a small interfering RNA comprising 19 to 29 nucleotides that are substantially complementary to 19 to 29 nucleotides of human SLC30A1. Human SLC30A1 has the sequence of SEQ ID NO: 3.

The methods described herein are particularly useful in cell culture applications. Thus, in one embodiment, the red blood cell precursors are cultured hematopoietic precursor cells or cultured precursor pluripotent stem cells. Hematopoietic precursor cells give rise to blood cells through the process of hematopoiesis. Typically, the source of hematopoietic precursor cells is either bone marrow or mobilized peripheral blood. Pluripotent stem cells are master stem cells that can give rise to all different cell types, including red blood cells. Once differentiated into mature red blood cells, the mature red blood cells can be administered, e.g., transplanted, to subjects, e.g., human subjects, in need thereof.

In another aspect, the method comprises administering a zinc chelator or an inhibitor of a zinc importer protein, e.g., SLC39A8; and wherein the administering inhibits survival of the red blood cell precursors, inhibits terminal differentiation of the red blood cell precursors to mature red blood cells, or a combination thereof.

As used herein, the term “zinc chelator” refers to any substance that is able to chelate a zinc (Zn²⁺) ion and thus deplete zinc from aqueous environments. Exemplary zinc chelators include (N,N,N′,N′-Tetrakis(2-Pyrimidyl)ethylenediamine) (TPEN), 10-phenanthroline, ethylene glycol tetraacetic acid (EGTA), diethyldithiocarbamate (DEDTC), ethylenediamine-N,N′-diacetic acid (EDDA), diethylenetriamine pentaacetic acid (DTPA), pyridine disulfide ethylenediaminetetraacetic acid (PSDE), amino-iminodiacetic acid (IDA), and 2-amino-4-fluorophenol N,N,O triacetic acid (AFTA), a zinc-binding peptide, or a combination thereof.

In an aspect, the inhibitor of the zinc importer protein is a therapeutic RNA, e.g., SEQ ID NO 3.

SEQ ID NO: 4
TGATAATTGTCACTGACAG (Mouse)

In an aspect, the inhibitory RNA is a small interfering RNA comprising 19 to 29 nucleotides that are substantially complementary to 19 to 29 nucleotides of human SLC39A8. Human SLC39A8 has the sequence of SEQ ID NO: 5. In an aspect, inhibiting survival of the red blood precursors further comprises removing the undifferentiated precursors from red blood cells terminally differentiated in vitro to provide red blood cells completely matured for transplantation therapy. Delaying differentiation can ensure the generation of billions of erythrocytes daily without precursor depletion or inadequate progeny genesis, both of which would yield anemia.

In another embodiment, a method of treating a patient suffering from a disease associated with defective erythropoiesis comprises administering an effective amount of a composition comprising zinc, a composition comprising an inhibitor of a zinc exporter protein, or a combination thereof;
wherein the administering promotes survival of red blood cell precursors, promotes terminal differentiation of red blood cell precursors to mature red blood cells, or a combination thereof;
or
administering an effective amount of a composition comprising a zinc chelator, a composition comprising an inhibitor of a zinc importer protein, or a combination thereof; wherein the administering inhibits survival of red blood cell precursors, inhibits terminal differentiation of red blood cell precursors to mature red blood cells, or a combination thereof.

Diseases associated with defective erythropoiesis include myelodysplastic syndromes (MDS), sickle cell anemia, or an anemia that is unresponsive to an erythropoiesis stimulating agent, e.g., erythropoietin. In this embodiment, the method comprises administering zinc or an inhibitor of a zinc exporter protein, e.g., SLC30A1.

In an embodiment, the composition comprising zinc, and/or the composition comprising an inhibitor of a zinc exporter protein is co-administered with a chemotherapeutic agent.

Exemplary chemotherapeutic agents include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, olaparib, ormnaplatin, oxaliplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, rucaparib, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, velaparib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, zorubicin, other PARP inhibitors, and combinations comprising at least one of the foregoing.

In another embodiment, the disease associated with defective erythropoiesis is leukemia. In this embodiment, the method comprises administering a zinc chelator or an inhibitor of a zinc importer protein, e.g., SLC39A8.

In an embodiment, the zinc chelator or inhibitor of a zinc importer protein is co-administered with a chemotherapeutic agent.

In another embodiment, a method of treating a human subject in need of a red blood cell transplant comprises
ex vivo culturing of hematopoietic precursor cells or precursor pluripotent stem cells in the presence of a composition comprising zinc, a composition comprising an inhibitor of a zinc exporter protein, e.g., SLC30A1, or a combination thereof; wherein contacting either promotes survival of hematopoietic precursor cells or precursors from pluripotent stem cells, or promotes terminal differentiation of the hematopoietic precursor cells or precursors from pluripotent stem cells to mature red blood cells;
isolating mature, differentiated red blood cells from the ex vivo culture; and
transplanting the mature, differentiated red blood cells into the human subject.

Exemplary human subjects in need of transplanted mature, differentiated red blood cells include patients of the disease associated with defective erythropoiesis.

Also included herein is a composition comprising a therapeutic RNA such as a small interfering RNA, the small interfering RNA comprising 19 to 29 nucleotides that are substantially complementary to a sequence of 19 to 29 nucleotides of SLC30A1 or SLC39A8. In one aspect, the small interfering nucleic acid comprises 19 to 29 nucleotides that are substantially complementary to a sequence of 19 to 29 nucleotides of SLC30A1 or SLC39A8. Exemplary small interfering RNAs are siRNA and shRNA.

As used herein, the term substantially complementary means that the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95% identical. Optimal alignment of sequences for comparisons of identity may be conducted using a variety of algorithms known in the art. Sequence identity may also be determined using the BLAST algorithm.

Further included are pharmaceutical compositions comprising a pharmaceutically acceptable excipient and a small interfering RNA, the small interfering RNA comprising 19 to 29 nucleotides that are substantially complementary to a sequence of 19 to 29 nucleotides of SLC30A1 or SLC39A8.

The invention is further illustrated by the following non-limiting examples.

### EXAMPLES

### Methods

Cell culture: G1E-ER-GATA-1 cells were cultured in Iscove's modified Dulbecco's medium (IMDM; Gibco) containing 15% FBS (Gemini), 1% penicillin-streptomycin (Gemini), 2 U/ml erythropoietin (Amgen), 120 nM monothioglycerol (Sigma), and 0.6% conditioned medium from a Kit ligand-producing CHO cell line, and 1 μg/ml puromycin (Gemini). ER-GATA-1 activity was induced by adding 1 μM β-estradiol (Steraloids) to media. To rescue heme synthesis, 1 mM 5-aminolevulinic acid hydrochloride (5-ALA; Sigma) was added to media.

Primary erythroid precursors from E14.5 murine fetal livers were cultured in StemPro®-34 (Gibco) supplemented with 10% nutrient supplement (Gibco), 1x L-glutamine (Cellgro), 1% penicillin-streptomycin (Gemini), 0.1 mM monothioglycerol (Sigma), 1 μM dexamethasone (Sigma), 0.5 U/ml erythropoietin (Amgen), and 1% conditioned medium from a Kit ligand-producing CHO cell line (expansion culture). To chelate zinc, 10 μM N,N,N′,N′-Tetrakis(2-pyridylmethyl)ethylenediamine (TPEN, Sigma, P4413) was added to the media, and 20 μM ZnCl₂ (Fisher Scientific) was added back. After two days, cells were differentiated in IMDM (Gibco) containing 10% FBS (Gemini), 10% plasma-derived serum (PDS; Animal Technologies), 5% protein-free hybridoma medium II (PFHM II; Gibco), 1% penicillin-streptomycin (Gemini), 0.1 mM monothioglycerol (Sigma) and 6 U/ml of erythropoietin (Amgen).

Primary human mononuclear cells were isolated from G-CSF-mobilized peripheral blood using Histopaque® (Sigma). Cells were maintained in StemSpan™ SFEM medium (Stem Cell Technologies) supplemented with 1× CC100 cytokine mix (Stem Cell Technologies) for 4 days. To induce differentiation, cells were cultured in basal differentiation media [IMDM containing 15% FBS, 2 mM L-glutamine, 1% BSA (Sigma), 500 μg/ml holo human transferrin (Sigma), and 10 μg/ml insulin (Sigma)] supplemented with 11 μM dexamethasone (Sigma), 1 μM β-estradiol (Steraloids), 5 ng/ml IL-3, 100 ng/ml SCF, and 6 U/ml erythropoietin (Amgen) for 5 days, followed by four days in basal differentiation media supplemented with 50 ng/ml SCF and 6 U/ml erythropoietin and then seven days in basal differentiation media containing 2 U/ml erythropoietin.

qRT-PCR. Total RNA was purified from 0.4-2×10⁶ cells with TRIzol® (Life Technologies). RNA was treated with DNase I (Life Technologies) for 15 min at room temperature. DNase I was inactivated by EDTA and heating at 65° C. for 10 min. DNase I-treated RNA was incubated with 125 ng of a 5:1 mixture of oligo (dT) primers and random hexamer at 68° C. for 10 min. RNA/primers were incubated with Moloney MLV reverse transcriptase (Life Technologies), 10 mM DTT (Life Technologies), RNAsin® (Promega), and 0.5 mM deoxynucleoside triphosphates at 42° C. for 1 h, and then heat inactivated at 98° C. for 5 min. Real-time PCR reactions were conducted with Power SYBR® Green Master Mix (Applied Biosystems) using ViiA™ 7 Real-Time PCR system (Applied Biosystems). Primer sequences are described in Table 1.

TABLE 1

| Primers for | | SEQ | |
|---|---|---|---|
| qRT-PCR | Species | ID NO: | Sequence (5′→3′) |
| Slc30a1 | mouse | 6 | caacaccagcaattccaacg |
| | | 7 | tgtcagactcctggatgagattc |
| Slc39a8 | mouse | 8 | gccagctgcacttcaacca |
| | | 9 | agagaagttcgagctggttatctg |
| Hbb-b1 | mouse | 10 | tttaacgatggcctgaatcactt |
| | | 11 | cagcacaatcacgatcatattgc |
| 18s | mouse/human | 12 | cgccgctagaggtgaaattct |
| | | 13 | cgaacctccgactttcgttct |
| HBB | human | 14 | tcctgaggagaagtctgccgt |
| | | 15 | ggagtggacagatccccaaag |
| HBA-1 | human | 16 | gggtggacccggtcaactt |
| | | 17 | gaggtgggcggccagggt |
| SLC30A1 | human | 18 | ccaataccagcaactccaacg |
| | | 19 | tacttccactgtatcaccacttctg |
| SLC39A8 | human | 20 | gccagctgcacttcaacca |
| | | 21 | gtaagactgctggacagatgacag |
| ACTB | human | 22 | ggccaaccgcgagaagat |
| | | 23 | ccagaggcgtacagggatagc |

Heme quantitation: Cells (2.8-3.9×10⁶) from two biological replicates per treatment were resuspended in cold hypotonic buffer (8.1 mM Na₂HPO₄, pH 7.4, 10 mM NaCl) and lysed via three successive rounds of freezing at −78° C., followed by thawing on ice. Cell lysates were centrifuged at 17,000×g for 30 min at 4° C., and supernatants were centrifuged at 100,000×g for 60 min at 4° C. Electronic absorption spectra of cleared lysates were recorded on a Varian Cary 4 Bio spectrophotometer, and the oxyhemoglobin concentration of each cleared lysate was calculated using extinction coefficients at 542 nm and 577 nm for human oxyhemoglobin monomers. Total heme in each cleared lysate was determined for two technical replicates using the pyridine hemochrome assay in which 200 μl of a stock solution of 0.1 N NaOH and 33% (v/v) pyridine was combined with 100 μl of the cleared lysate. Upon addition of 2-5 mg of sodium dithionite, electronic absorption spectra were recorded, and total heme concentrations were calculated using known extinction coefficients of the ferrous bis(pyridine) protoheme complex at 525 nm and 556 nm as is known in the art.

Flow cytometry. For intracellular zinc quantitation in mouse cells, cells were washed twice with PBS and incubated with 1 μl/ml Ghost Dye™ Red 780 (TONBO Biosciences) for 30 min at 4° C. Cells were washed once with PBS containing 2% FBS and twice with HBSS and treated with HBSS containing 1 μM FluoZin™-3 AM (Invitrogen), 0.02% Pluronic® F-127 (Invitrogen), BV421-conjugated anti-mouse CD71 (0.2 μg in 100 μl, Biolegend, 113813), and APC-conjugated anti-mouse Ter119 (0.2 μg in 100 μl, Biolegend, 116212) for 30 min at room temperature. Cells were washed once with HBSS and incubated for 15 min at room temperature. Cells were then washed twice with PBS and analyzed. To distinguish enucleated cells from nucleated erythroblasts, cells stained with Ghost Dye™ Red 780 were incubated with 2 μg/ml thiazole orange (Sigma, 390062) in PBS for 40 min at room temperature. Cells were washed once with PBS and stained with BV421-conjugated anti-mouse CD71 and APC-conjugated anti-mouse Ter119 for 30 min at 4° C. Cells were then washed twice with PBS and analyzed. For intracellular zinc quantitation in human cells, cells were washed twice with HBSS and incubated with HBSS containing 1 μM FluoZin™-3 AM, 0.02% Pluronic® F-127, PE-conjugated anti-human CD71 (0.024 μg in 100 μl, eBioscience, 12-0719-42), and APC-conjugated anti-human CD235a (0.006 μg in 100 μl, eBioscience 17-9987-42) for 30 min at room temperature. Cells were washed once with HBSS and incubated for 15 min at room temperature. Cells were then washed twice with PBS, stained with DAPI, and analyzed. Samples were analyzed using a BD LSR II (BD Biosciences).

Primary murine erythroid precursor cell isolation. E14.5 murine fetal liver cells were incubated with biotin-conjugated anti-mouse CD3c (3 μl/ml, Biolegend, 100304), anti-mouse/human CD11b (3 μl/ml, Biolegend, 101204), anti-mouse CD19 (3 μl/ml, Biolegend, 115504), anti-mouse/human CD45R (3 μl/ml, Biolegend, 103204), anti-mouse GR-1 (3 μl/ml, Biolegend, 108404), anti-mouse CD71 (3 μl/ml, Biolegend, 113803), and anti-mouse Ter119 (5 μl/ml, Biolegend, 116204) for 15 min at 4° C. Cells were washed with PBS containing 2% FBS, 2.5 mM EDTA, and 10 mM glucose. Cells were incubated with MojoSort™ Streptavidin Nanobeads (75 μl/ml, Biolegend, 480016) for 15 min at 4° C. Cells were washed with PBS containing 2% FBS, 2.5 mM EDTA, and 10 mM glucose. Cells were resuspended in 3 ml PBS containing 2% FBS, 2.5 mM EDTA, and 10 mM glucose and incubated for 6 min with a magnet. Unbound cells were collected.

shRNA-mediated knockdown. shRNAs were cloned into MSCV-PIG vector (from Dr. Mitchell Weiss) using Bgl II and Xho I sites. Sequences of oligonucleotides used to construct shRNA plasmids are described in Table 1. shRNA-PuroR-IRES fragment was ligated into pMSCV-IRES-mCherry FP (Addgene, #52114) using Bgl II and Nco I sites. 293T cells transfected with 15 μg of pMSCV-PuroR-IRES-mCherry vector and pCL-Eco packaging vector produced retrovirus expressing shRNA. Cells were added to 100 μl viral supernatant, polybrene (8 μg/ml), and HEPES buffer, and spinoculated at 2,600 rpm for 90 min at 30° C.

Preparing microscope slides. 0.5×105 (less mature cells) or 4×105 cells (more mature cells) in 200 μl PBS were centrifuged at 280 rpm or 250 rpm, respectively, for 10 min using cytospin. The slides were stained with Wright-Giemsa Stain (Sigma, WG16-500ML).

Protein digestion and isolation. Tissue samples were suspended in 100 ρl guanidinium chloride (6 M) and homogenized with a cup horn sonicator (Qsonica) until fully in solution. Protein was precipitated in 900 μl methanol and pelleted (5,000×g, 15 min). Pellets were resuspended in 500 μl lysis buffer (8 M urea, 100 mM tris pH 8, 10 mM TCEP, 40 mM 2-chloroacetamide) and diluted to >1.5 M urea with 50 mM tris pH 8. Trypsin was added (1:50::g:g) and incubated overnight at ambient temperature with gentle rocking. Additional trypsin (1:200:g:g) was added in the morning and after an additional hour, peptides were isolated and desalted with Strata®-X polymeric reverse phase resin (Phenomenex). Peptide yield was calculated by bicinchoninic acid (BCA) assay (Thermo Pierce).

High pH reverse-phase fractionation. Samples were resolved with an Acquity BEH C18 reverse phase column at 60° C. (130 A pore size, 1.7 μm particle size, 2.1×100 mm, Waters Corp) using a 1260 Infinity liquid chromatograph (600 μl/min flow rate, Thermo Scientific). Mobile phase A consisted of 20 mM ammonium bicarbonate in H2O. Mobile phase B consisted of 20 mM ammonium bicarbonate in ACN/H2O. Elution was achieved with a gradient, and 8 fractions were collected from 5-23 min with concatenation of fraction 1 and 9, 2 and 10, . . . 8 and 16.

LC-MS/MS analysis. Concentrated fractions of sample four were resuspended in 16 μl 0.2% formic acid. All other samples were resuspended to 1 μg/μl. Each sample (2 μl) was analyzed on a Q-LTQ-OT mass spectrometer (Thermo Scientific Fusion Lumos) following LC separation. Injections were made onto a 75-360 μm inner-out diameter fused silica capillary column with a laser-pulled electrospray tip packed with BEH C18 (130A pore, 1.7 μm particle size, 35 cm, Waters Corp) with a Dionix Ultimate 3000 uHPLC. Peptides were resolved on a gradient, and eluted peptides entered the mass spectrometer following positive mode electrospray ionization. MS1 survey scans were performed in the orbitrap (240K resolution, AGC target −1e6, 50 ms max injection time). MS2 analysis of HCD-generated (30% NCI) fragment ions were performed in the ion trap (turbo resolution, AGC target −3e4, 11 ms max injection time). Monoisotopic precursor selection and dynamic exclusion (15 s) were enabled.

Data analysis. Thermo RAW files were processed with the MaxQuant quantitative software suite. Searches were performed with precursor mass tolerance of 50 ppm and product ion mass tolerance of 0.2 Da. Carbamidomethylation of cysteines was imposed as a fixed modification. Oxidation of methionines was set as a variable modification. ‘Match between runs’ and ‘label free quantitation’ were enabled using a match time window of 0.7 min and minimum ratio count of 1. Search results were filtered to a false discovery rate of 1%.

Statistics and reproducibility. No data were excluded from the analysis. Enumeration of nucleated and enucleated erythroblasts was conducted by a blinded analysis. All attempts of replication were successful. At least two independent experiments were conducted with at least two biological replicates to collect sample sizes allowing for quantitative comparisons. Representative images from these experiments are shown. Exact numbers of sample sizes and independent experiments are stated in the corresponding figure legends. Statistical analyses were performed with GraphPad Prism 7. Data are presented as stated in the respective figure legends. To compare the difference between two groups, unpaired or paired two-tailed t-tests were performed. For multiple comparisons, one-way ANOVA was conducted with Dunnett's test or Tukey's test.

### Example 1: Gata/Heme Multi-Omics

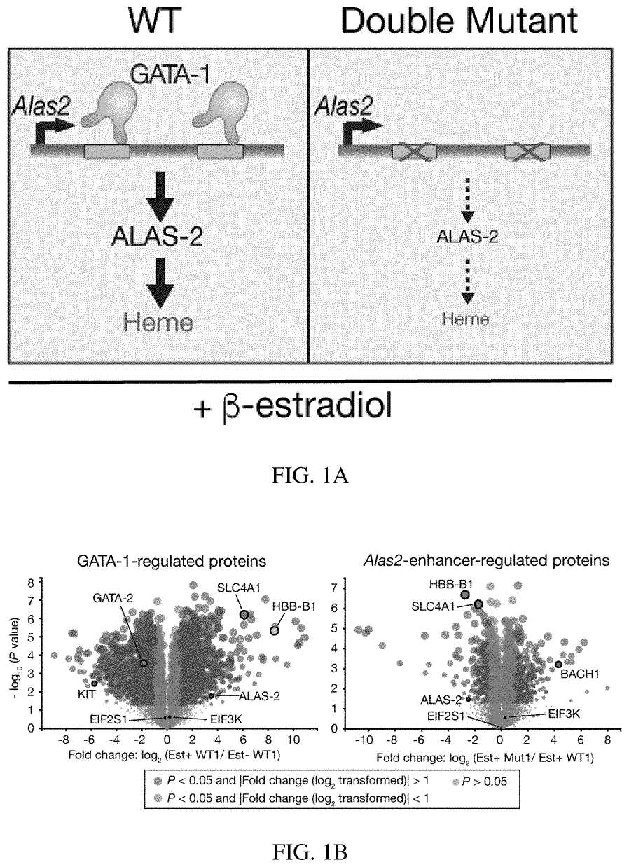

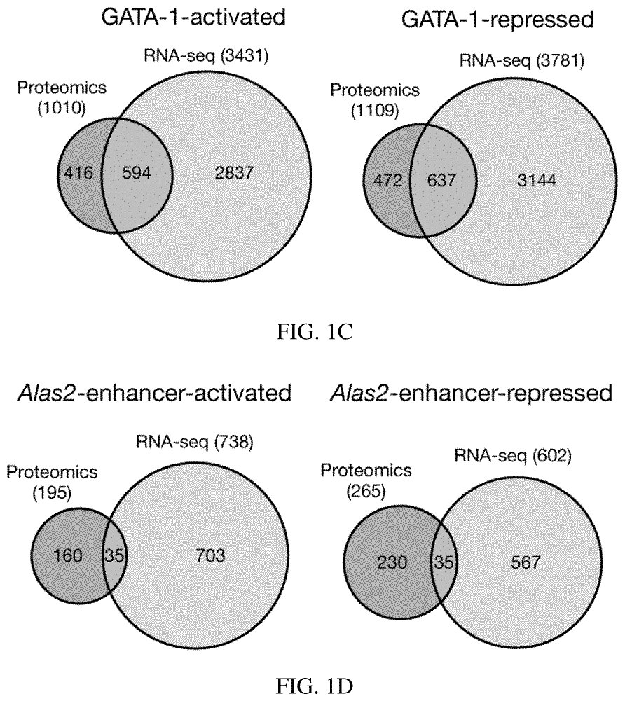

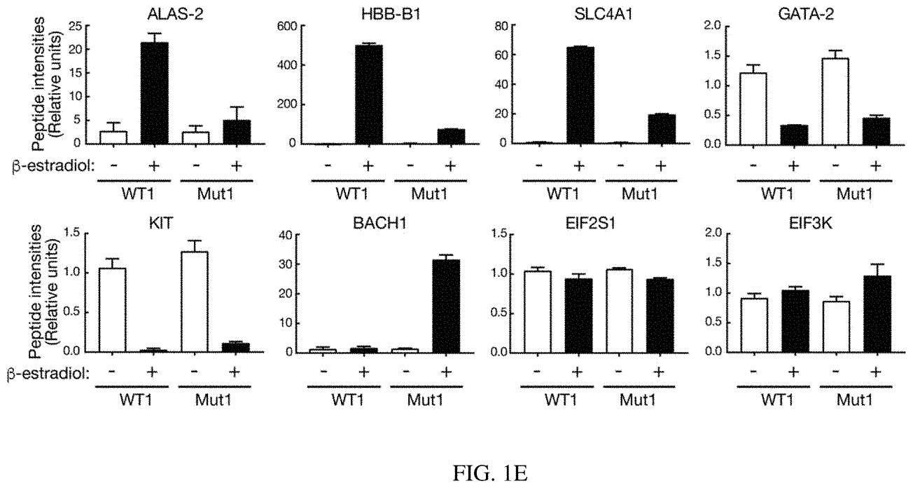

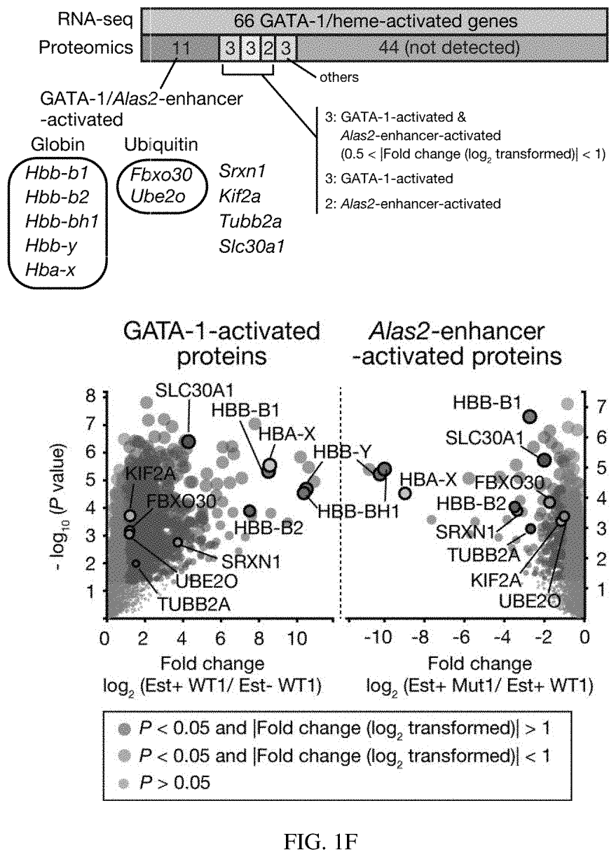

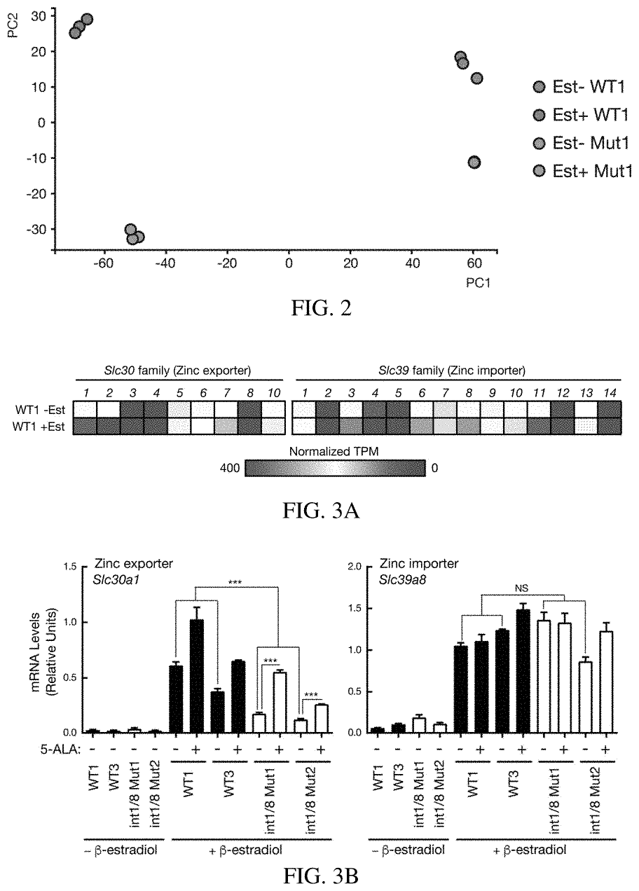

To decipher how GATA factor and heme mechanisms intersect and function independently, high-resolution mass spectrometry was utilized to measure global protein abundances in a system that allows GATA-1 genetic complementation in cells with normal and subphysiological heme levels (FIGS. 1A and 2). Heme was rendered approximately 30-fold lower by genetic editing of GATA motifs in Alas2 enhancers in G1E-ER-GATA-1 cells. Nanoflow LC-MS/MS analysis of complex peptide mixtures, generated by digestion of the whole proteomes with trypsin, was performed using a quadrupole-Orbitrap™-dual cell linear ion trap hybrid mass spectrometer. Raw mass spectral data were searched with the MaxQuant proteomics software package against a database of mouse proteins concatenated with reverse sequences as decoy targets as is known in the art. The analysis yielded 1,069,305 peptide spectral matches, 111,385 unique peptide identifications, and 7,168 detected proteins (1% false discovery rate). Label-free quantitation, also calculated with MaxQuant, yielded quantitative data for 6,017 proteins across all samples.

The GATA-1-regulated sector of the erythroblast proteome was determined by comparing uninduced and β-estradiol-treated G1E-ER-GATA-1 cell proteomes (FIG. 1B). This analysis revealed 1010 and 1109 GATA-1-upregulated and -downregulated proteins (p<0.01), respectively (FIG. 1C and Table 1). To identify co-regulated mRNAs and proteins, the results were compared with RNA-seq data. GATA-1 upregulated and downregulated 594 and 637 mRNAs/proteins, respectively (Data not shown).

To establish the heme contribution to GATA-1-regulated and -insensitive proteome sectors, β-estradiol-treated G1E-ER-GATA-1 cells and Alas2 enhancer mutant G1E-ER-GATA-1 cells were compared (FIG. 1B). Differentially expressed proteins (>2-fold) conforming to each regulatory mode were parsed into upregulated and downregulated cohorts. Of the Alas2-enhancer-upregulated (195) and -downregulated (265) proteins, 35 and 35, respectively, of the mRNAs were regulated (FIG. 1D and data not shown).

GATA-1/Alas2-enhancer-induced genes and proteins included the erythroid genes Hbb-b1 and Slc4a1 (FIG. 1E). GATA-1-mediated repression of the well-characterized repressed genes Gata2 and Kit and their encoded proteins were insensitive to Alas2 enhancer mutations. Whereas Bachl mRNA was induced in β-estradiol-treated WT and double-mutant cells, Bachl protein increased only in β-estradiol-treated double-mutant cells (FIG. 1E), highlighting the post-translational control of Bachl levels. The prior RNA-seq analysis identified 66 GATA-1/Alas2 enhancer-co-regulated genes. Eleven of the encoded proteins [5 hemoglobin subunits, 2 ubiquitination components (Fbxo30 and Ube2o), Srxn1, Kif2a, Tubb2a and the zinc exporter Slc30a1] were GATA-1/Alas2-enhancer-upregulated (FIG. 1F). Of the remaining 55 GATA-1/Alas2 enhancer-activated genes, 11 of the encoded proteins did not meet all criteria to be deemed GATA-1/Alas2 enhancer-co-regulated, and 44 were not definitively identified.

### Example 2: Gata/Heme Circuit Reconfigures Zinc Transport Machinery During Differentiation: Evidence for a Zinc Transporter Switch

As the multi-omic analysis revealed essential hemoglobin components and the critical regulator of late-stage erythroid maturation UBE2O, a ubiquitin-conjugating enzyme, it was considered whether proteins regulated similarly also have vital roles in erythrocyte biology. The zinc finger transcription factors GATA-1 and KLF1 control erythrocyte differentiation, and zinc is a critical cofactor of the heme biosynthetic enzyme delta-aminolevulinic acid dehydratase (ALAD). How these factors sense changes in environmental and intracellular zinc is unclear, and zinc transport machinery had not previously been linked to erythrocyte differentiation (or other types of differentiation) or to GATA factor networks.

The mammalian Solute Carrier (SLC) family consists of 9 zinc exporters [51c30 family, also termed ZnT (Zinc Transporter) family] and 14 zinc importers [51c39 family, also termed ZIP (Zrt-, Irt-like protein) family]. Slc30a1, Slc39a8, and Slc39a10 were detected in erythrocyte membrane fractions. The targeted deletion of murine Slc30a1 is embryonic lethal shortly after implantation. Slc39a8 deletion is lethal shortly after birth, and although the mutants are severely anemic, no mechanistic analyses were reported.

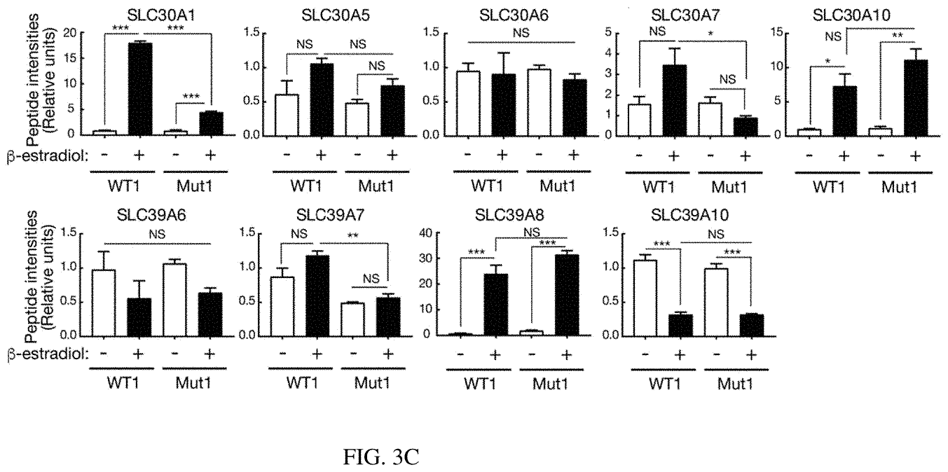

Database mining revealed differential expression of zinc exporters and importers during mouse and human hematopoiesis. To determine which exporters and importers were expressed and regulated, transcriptomes of untreated and β-estradiol-treated G1E-ER-GATA-1 cells were compared. Slc30a1 and Slc39a8 were the most highly expressed exporter and importer, respectively, and both were GATA-1-activated (FIG. 3A). qRT-PCR confirmed GATA-1 regulation in two wild type and double-mutant clones (FIG. 2B). As Slc30a1, but not Slc39a8, expression declined in heme-deficient double-mutant clones, these genes have shared (GATA-1-induced) and unique regulatory modes.

In principle, reduced heme may reversibly or irreversibly impact a process, and the Alas2 enhancer mutations might indirectly impact genes sharing the Alas2 subnuclear neighborhood. To rigorously ascribe heme regulation, we used the heme precursor 5-ALA to bypass ALAS-2 function and rescue heme biosynthesis. 5-ALA increased Slc30a1, but not Slc39a8, expression, providing further evidence for differential heme-regulation (FIG. 3B). The proteomics analysis detected 5 zinc exporters and 4 importers (FIG. 3C). Whereas Slc30a1 was GATA-1/Alas2-enhancer-induced, Slc39a8 was GATA-1-induced, but not Alas2-enhancer-regulated (FIG. 3C).

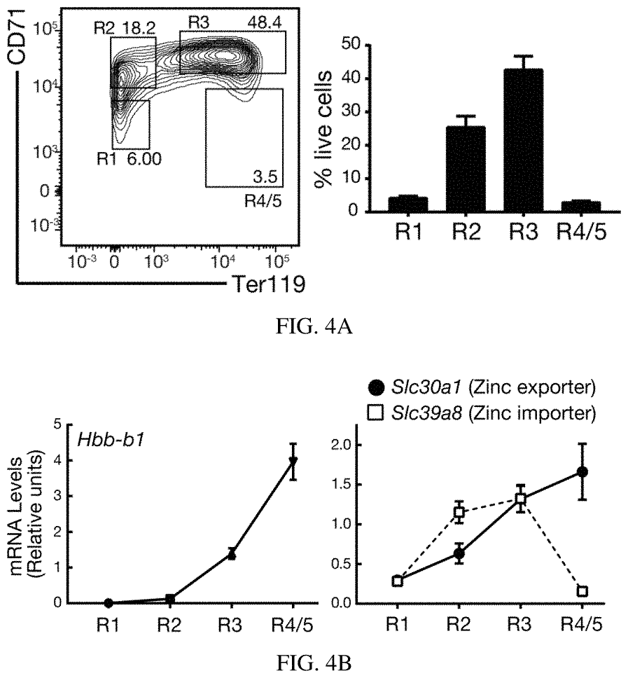

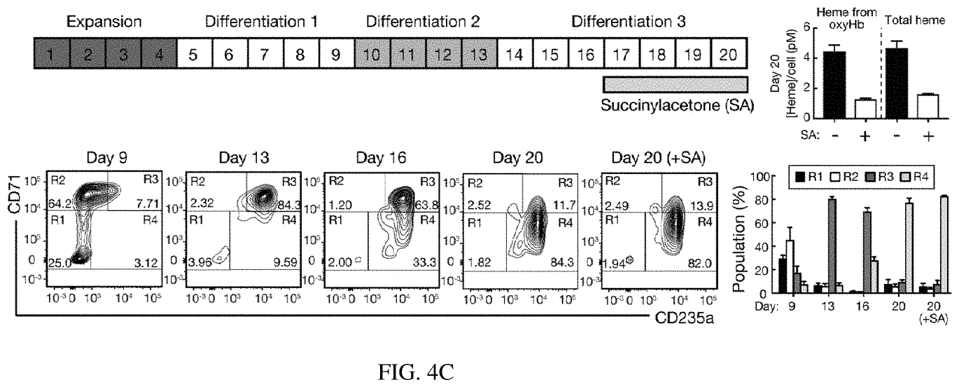

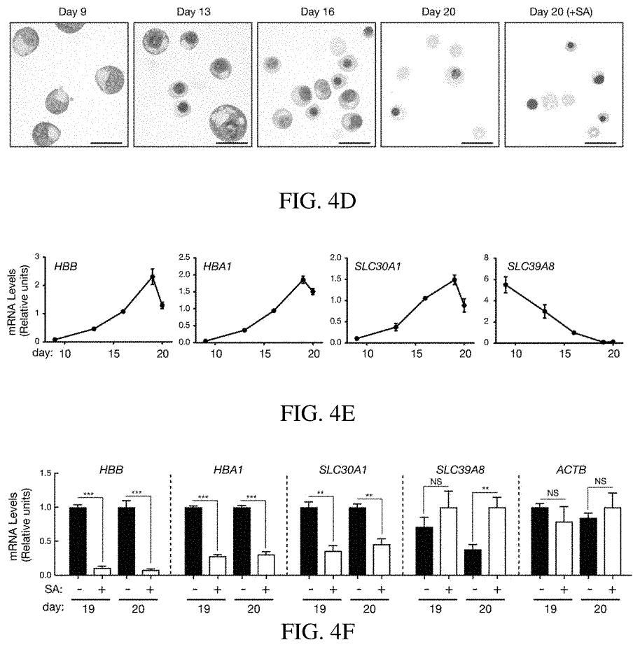

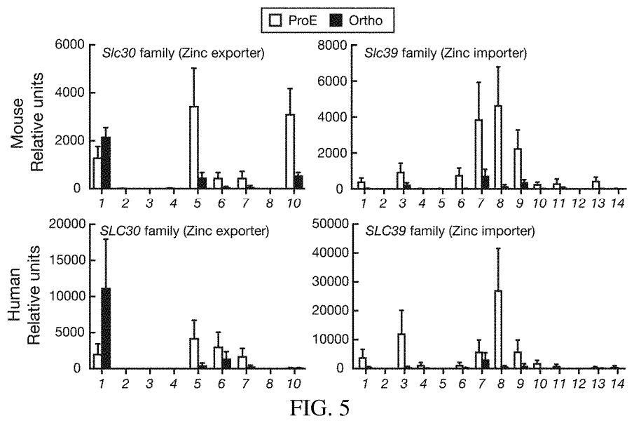

We then tested whether the zinc transporter genes Slc30a1 and Slc39a8 that share GATA-1 regulation and differ in heme regulation are controlled similarly in primary cells. Lineage-depleted hematopoietic precursors from E14.5 murine fetal livers were cultured for 72 hours, and flow cytometry was used to isolate cell populations based on erythroid surface marker (CD71 and Ter119) expression (FIG. 4A). Hbb-b1 expression increased as immature erythroid precursors (R1) matured into terminally differentiated reticulocytes and erythrocytes (R4/5) (FIG. 4B, left). Whereas Slc30a1 expression also increased, Slc39a8 increased during the initial differentiation, but was downregulated thereafter (FIG. 4B, right). Analysis of all zinc exporters and importers indicated that Slc30a1 was the predominant zinc exporter expressed in murine orthochromatic erythroblasts, while Slc39a8 was the predominant importer expressed in immature proerythroblasts (FIG. 5).

To test whether the zinc transporter expression pattern is evolutionarily conserved, a human system was utilized in which mononuclear cells from G-CSF-mobilized peripheral blood are cultured for four days and subjected to erythroid differentiation for 16 days (FIG. 4C). The erythroid maturation stage was quantified by flow cytometry with CD71 and CD235a surface markers (FIG. 4C, bottom). Wright-Giemsa staining revealed a major increase in mature erythroid cells at days 16 and 20 (FIG. 4D). Resembling the globin genes HBB and HBA1, SLC30A1 expression increased 14-fold during differentiation (FIG. 3E). SLC39A8 expression declined 59-fold during differentiation (FIG. 4E). Slc39a8 upregulation in murine G1E-ER-GATA-1 cells and fetal liver cells was not evident in the human system, since the day nine human culture already consisted of R1, R2, and R3 cells (FIG. 4C). Analysis of all zinc exporters and importers revealed SLC30A1 as the predominant zinc exporter expressed in human orthochromatic erythroblasts, and SLC39A8 as the predominant importer expressed in immature proerythroblasts (FIG. 5). Thus, the major zinc exporter and importer genes were upregulated early in erythroid differentiation, and the importer was downregulated during terminal differentiation in mouse and human cells.

To determine if SLC30A1 is heme-regulated in the human system, the heme biosynthesis inhibitor succinylacetone was used to reduce heme during the last four days of culture, which did not impact cellularity (FIGS. 4C and D). Succinylacetone decreased heme in oxyhemoglobin and total heme 3.4- and 2.9-fold, respectively, (FIG. 4C, right) and reduced SLC30A1 expression, as well as other heme-dependent GATA-1 target genes HBB and HBA1 (FIG. 4F). ACTB, encoding β-actin, was unaffected (FIG. 3F). These primary mouse and human cell studies revealed an evolutionarily conserved mechanism to differentially control zinc transporter genes during erythroid differentiation, culminating in a switch from importer and exporter expression to solely exporter expression.

### Example 3: Establishing/Maintaining Zinc Levels as a New Paradigm of Regulating Cellular Differentiation

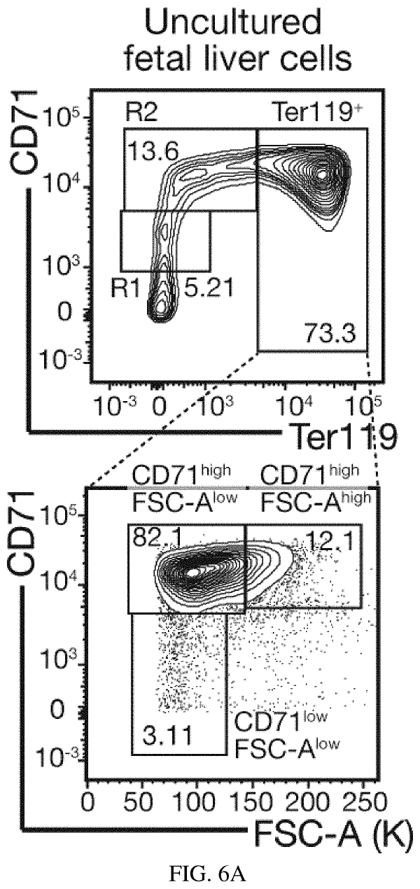

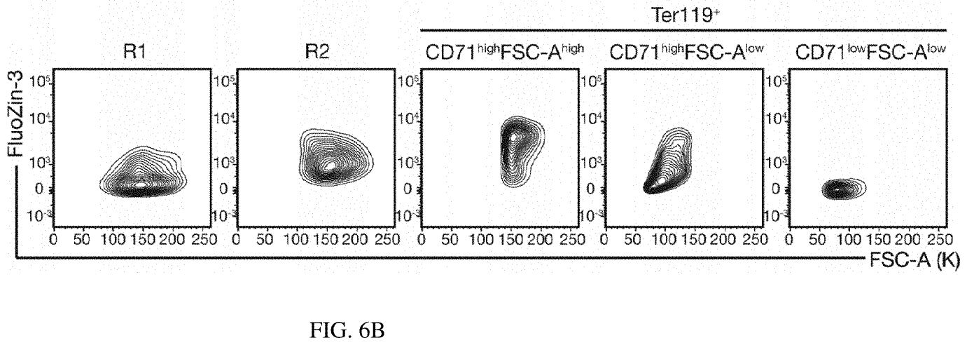

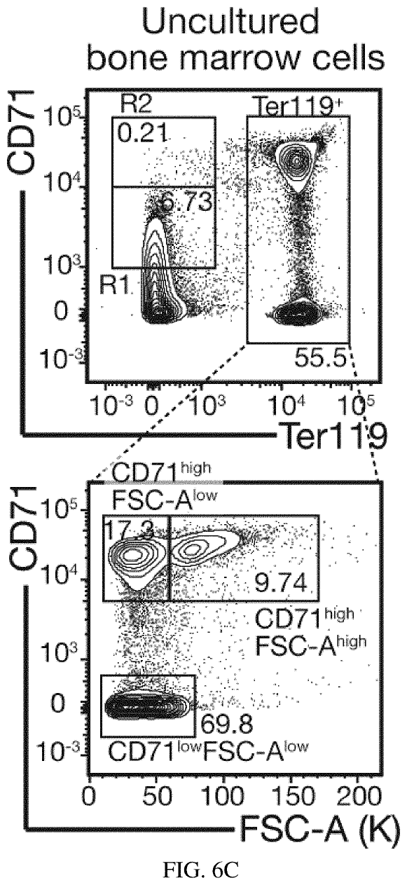

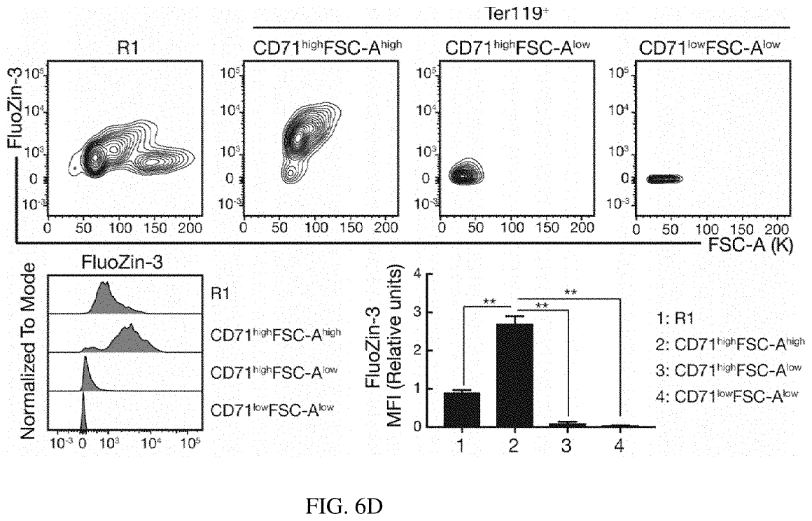

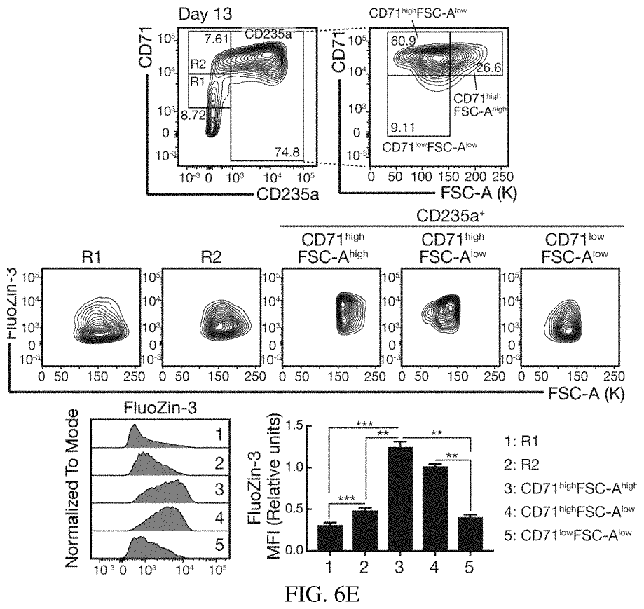

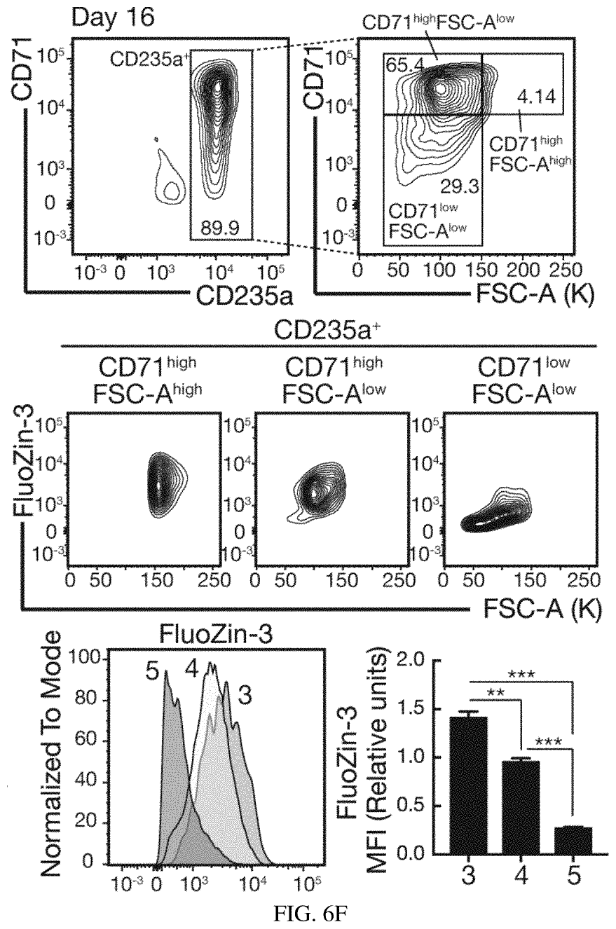

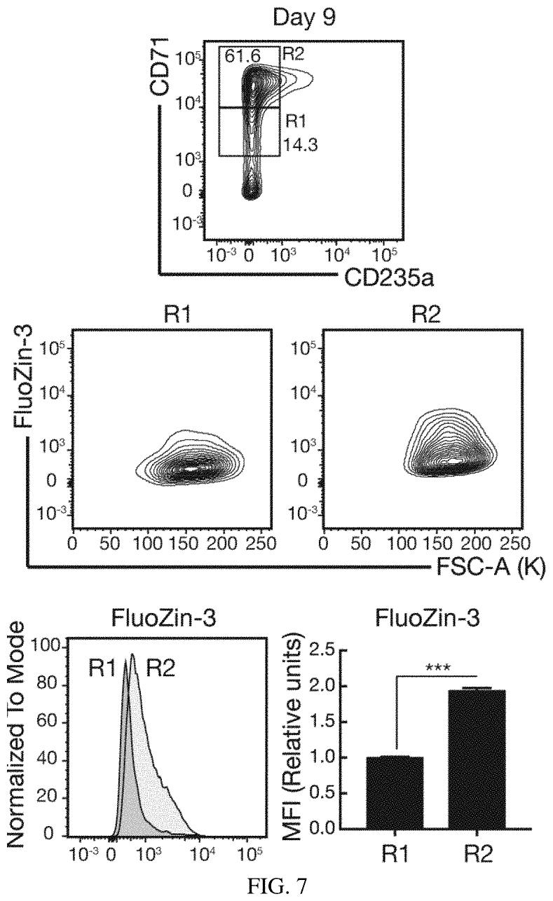

The GATA-1-regulation and differential heme-regulation of Slc30a1 and Slc39a8 suggested that intracellular zinc may constitute a previously unrecognized mechanism to control erythroid differentiation. To determine how the GATA/heme-instigated circuit involving a zinc transporter switch impacts zinc levels during erythroid maturation, the fluorescent zinc indicator FluoZin™-3 was used. Using a flow cytometric assay with uncultured E14.5 murine fetal liver cells, zinc, CD71 and Ter119were quantified (FIGS. 6A and B). Ter119+ cells were analyzed by gating for CD71highFSC-Ahigh (immature, large erythroblasts), CD71highFSC-Alow (more mature erythroblasts), and CD71lowFSC-Alow (mature erythroblasts, reticulocytes, and erythrocytes) populations. The FluoZin™-3 signal increased 8.4-fold during early maturation and decreased 70-fold thereafter (FIG. 6B), as predicted by the zinc transporter switch, in which expression of the zinc exporter and importer was sustained and decreased, respectively, during terminal differentiation. Similarly, in uncultured bone marrow, the FluoZin™-3 signal increased 3-fold during early erythroblast maturation and decreased 77-fold in more mature erythroblasts (FIGS. 6C and D). The rise in intracellular zinc during initial erythroid maturation, followed by a steep decline during terminal differentiation, was recapitulated in the primary human erythroblast culture system (FIGS. 6E and 7)

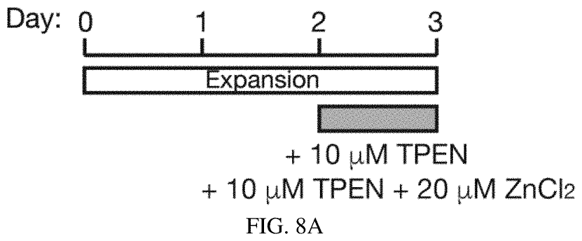

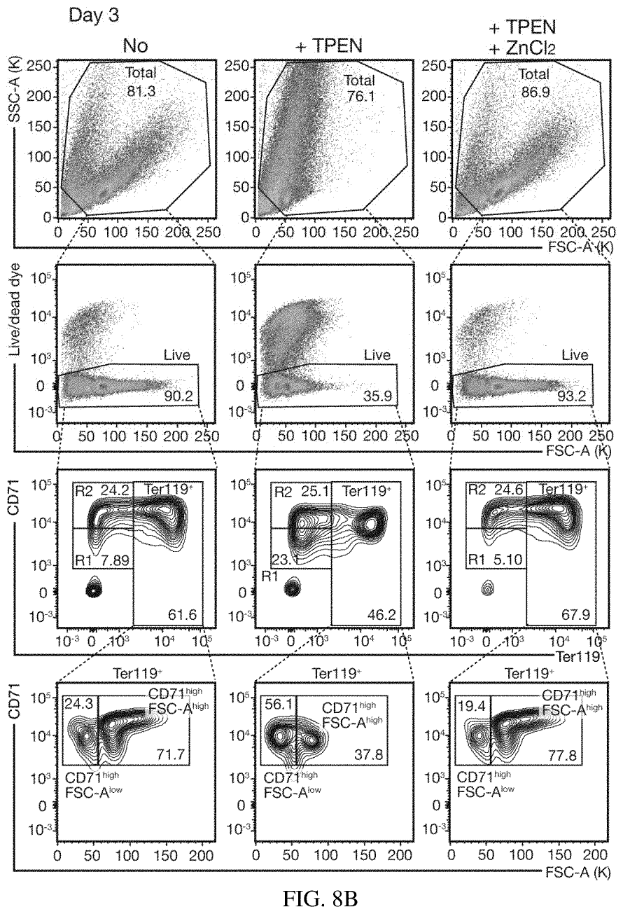

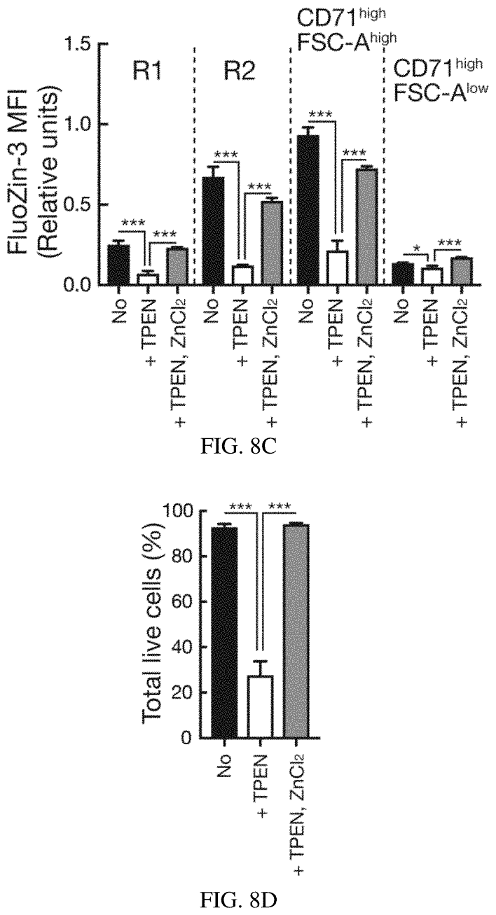

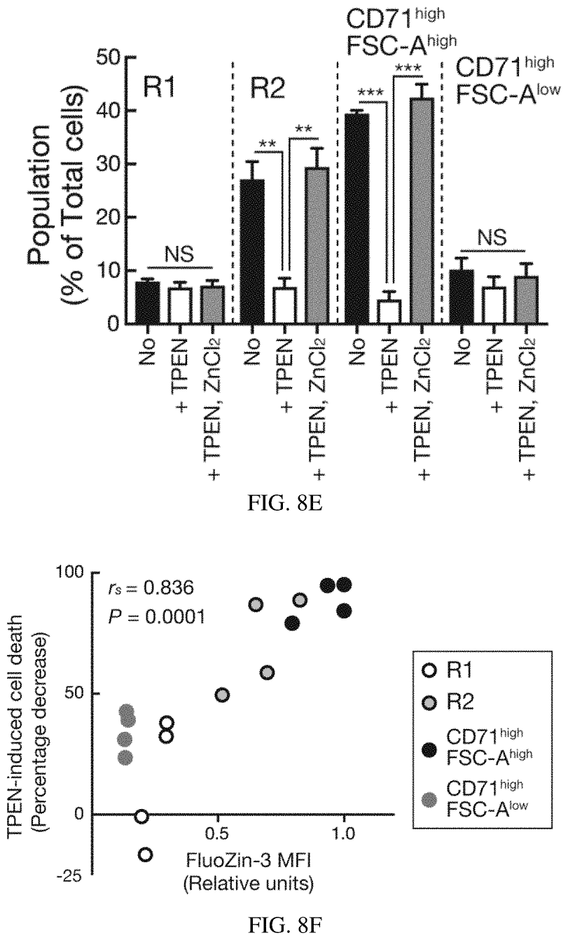

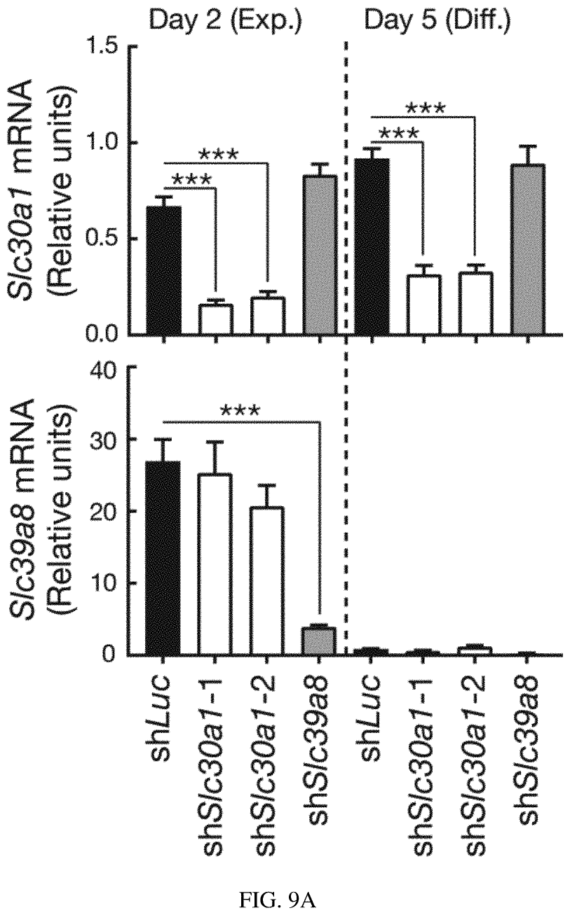

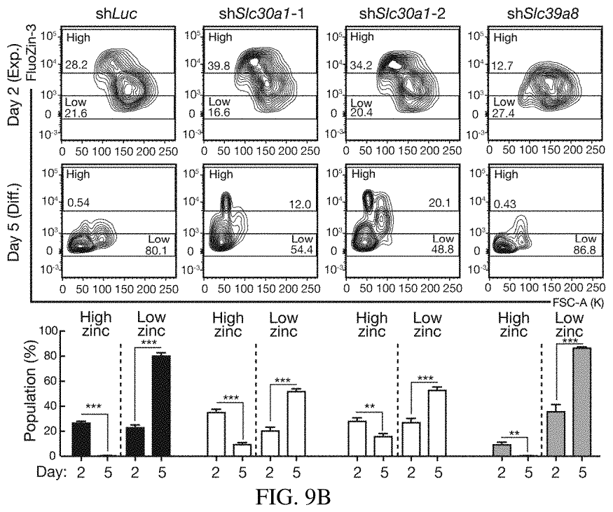

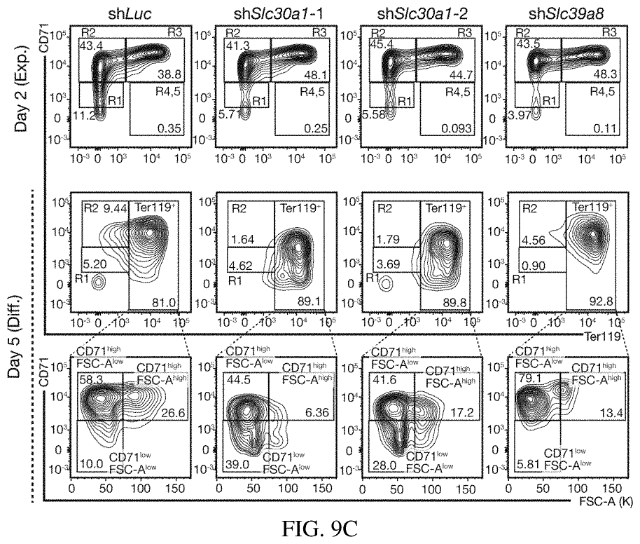

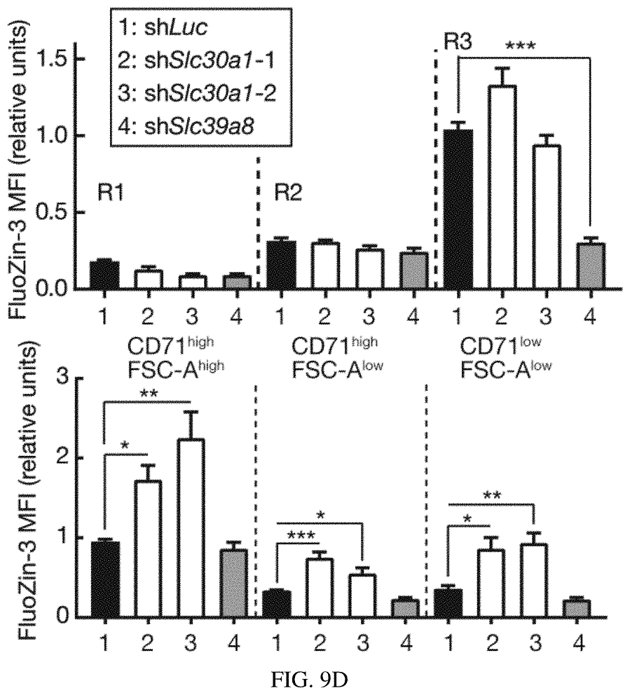

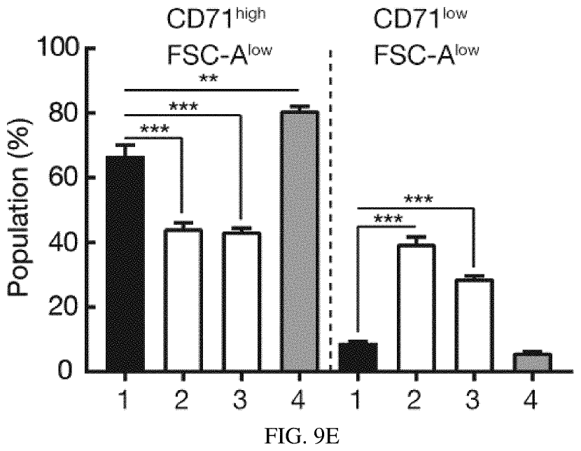

To analyze the consequences of the progressive rise in intracellular zinc, prior to the zinc transporter switch, the zinc chelator TPEN was used to reduce zinc levels in a culture of lineage-negative fetal liver-derived hematopoietic precursors (FIGS. 8A and B). TPEN reduced intracellular zinc levels in erythroid precursors of varying maturation stage (FIG. 8C) and considerably decreased the live cell population (FIG. 8D). TPEN decreased populations with the highest intracellular zinc levels (R2 and CD71highFSC-Ahigh) (FIG. 8E) and adding ZnCl2 to the media negated the actions of TPEN (FIGS. 8B-E). Intracellular zinc positively correlated with sensitivity to TPEN-induced cell killing (FIG. 9F), thus supporting a model in which GATA-1-mediated induction of the zinc importer Slc39a8 elevates intracellular zinc as a novel survival mechanism.

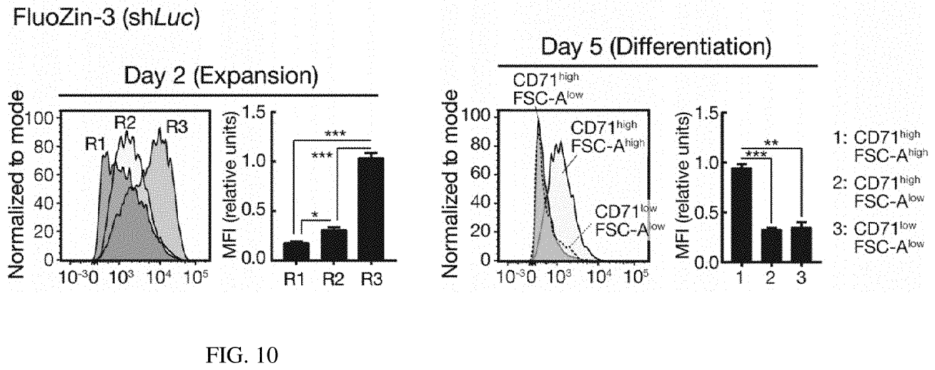

To test whether reconfiguring the mechanism governing intracellular zinc impacts differentiation and to elucidate Slc30a1 and Slc39a8 function, shRNA-based loss-of-function analyses were conducted. Lineage-negative hematopoietic precursors (E14.5) were expanded for two days or were expanded and then differentiated for three days. Cells were infected with retroviruses expressing shRNAs targeting Slc30a1, or Slc39a8 (Luciferase as a control), which downregulated Slc30a1 and Slc39a8 mRNA 73 and 86%, respectively, at day two and 65 and 71% at day five (FIG. 9A). The Slc30a1 loss did not impact Slc39a8 mRNA levels and vice versa. The high- and low-zinc populations in the cells expressing each shRNA decreased and increased, respectively, during differentiation (FIG. 9B). Cells were expanded for two days, CD71 and Ter119 were quantified, and populations were analyzed by gating for R1 (immature erythroblast), R2 (proerythroblast), and R3 (early and late basophilic erythroblasts) populations. The Ter119+ population generated by differentiation at day 5 included CD71highFSC-Ahigh, CD71highFSC-Alow and CD71lowFSC-Alow populations (FIG. 9C). Flow cytometric analysis of control cells expanded for two days revealed a 5.6-fold increase in intracellular zinc as R1 differentiated into R3 (FIG. 10). At day five, zinc was 2.8-fold higher in less mature CD71highFSC-Ahigh cells relative to the other populations (FIG. 10). These results resembled those with uncultured cells (FIGS. 6A and B).

In expansion culture, Slc39a8 downregulation decreased intracellular zinc 3.3-fold in R3 cells (FIG. 9D, top), demonstrating Slc39a8 function to establish and/or maintain intracellular zinc levels in immature erythroblasts. Whereas Slc30a1 mRNA decreased by 73% in the knockdown cells, the downregulation did not impact intracellular zinc in these cells in which intracellular zinc was already high (FIG. 9D, top).

In an erythroid differentiation culture, Slc30a1 downregulation increased intracellular zinc in all populations. In this context, Slc39a8 downregulation had no effect (FIG. 9D, bottom). Thus, Slc30a1 establishes and/or maintains intracellular zinc in more mature erythroblasts.

Within Ter119+ populations, Slc30a1 loss increased intracellular zinc 2.2-fold, decreased the CD71highFSC-Alow population 1.5-fold and increased the more mature CD71lowFSC-Alow population 3.9-fold. Strikingly, the terminally differentiated CD71lowFSC-Alow population constituted 35% of the total Ter119+ population (FIG. 9E). Downregulating Slc39a8 did not alter zinc in the more mature populations (FIG. 9D, bottom) and did not impact cellularity (FIG. 9E). Thus, corrupting the zinc exporter mechanism that restricts intracellular zinc in more mature erythroblasts increases intracellular zinc and promotes differentiation.

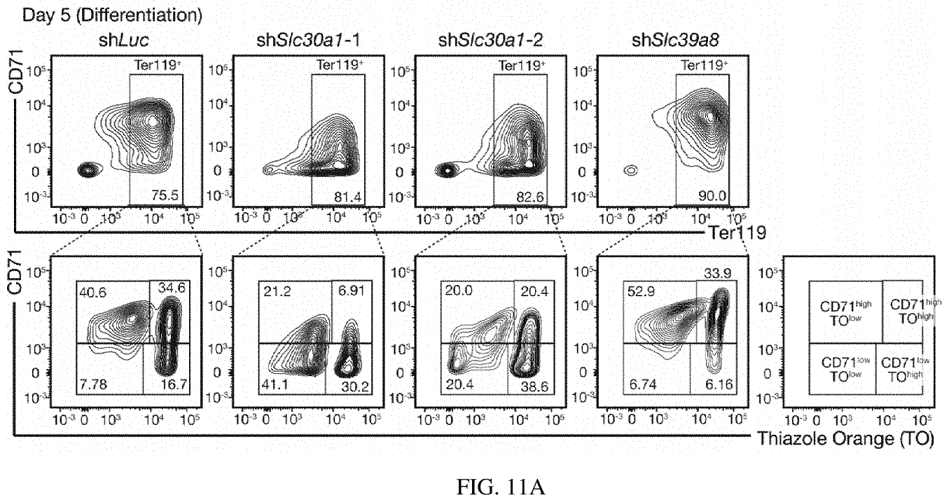

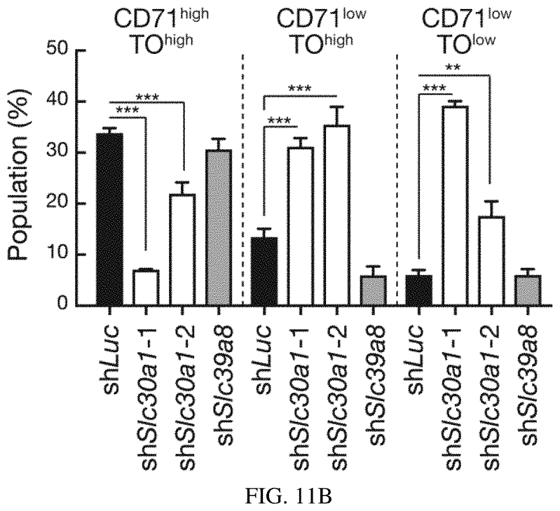

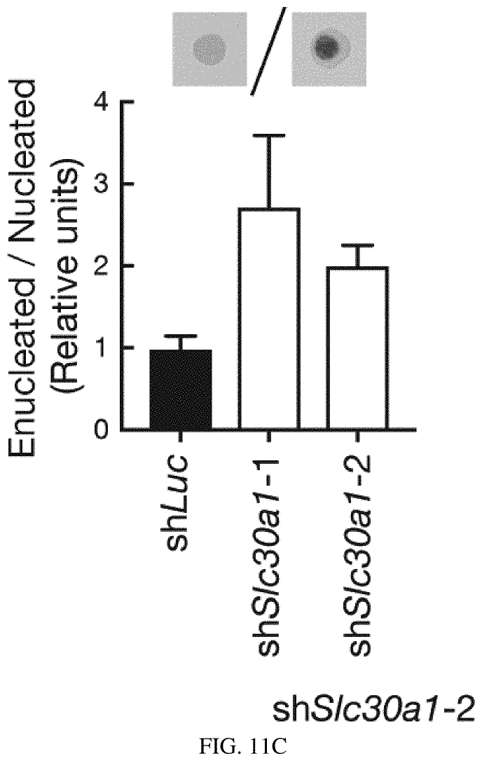

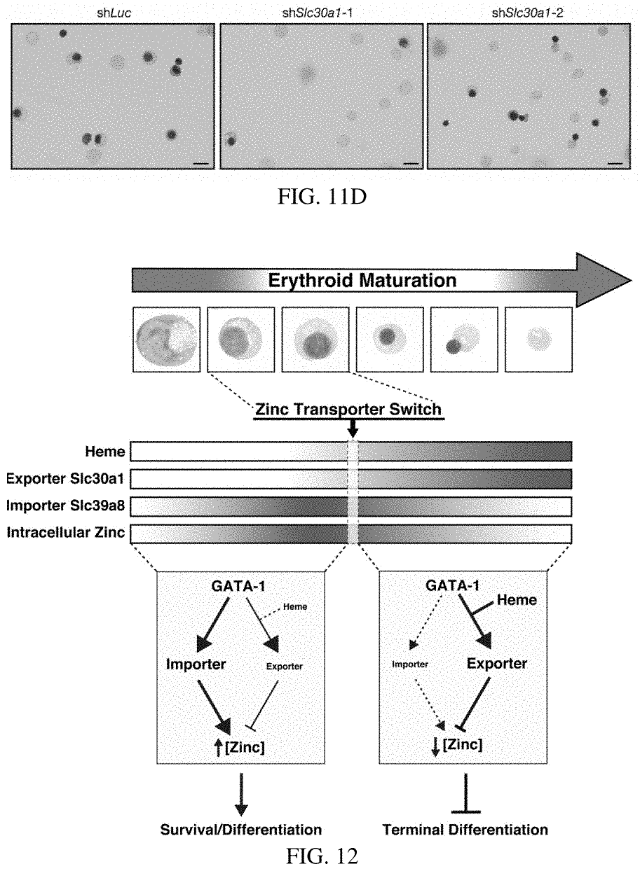

To further analyze how reconfiguring zinc transport machinery impacts differentiation, the DNA- and RNA-binding dye thiazole orange (TO) was used to discriminate between nucleated erythroblasts and enucleated progeny (FIG. 11A). Slc30a1 downregulation decreased the CD71highTOhigh population (nucleated erythroblasts) 2.3-fold, while increasing the CD71lowTOhigh (more mature nucleated erythroblasts) and CD71lowTOlow (enucleated reticulocytes and erythrocytes) cells 2.5 and 4.8-fold, respectively (FIGS. 11B-D). Slc39a8 downregulation did not influence the cell populations.

In aggregate, these results establish a paradigm in which GATA-1 induces a zinc importer that increases intracellular zinc early in erythroid differentiation to confer cell survival. As differentiation proceeds and heme increases, heme amplifies GATA-1-dependent induction of a zinc exporter, and importer expression is repressed. This GATA/heme-instigated zinc transporter switch expels intracellular zinc. As downregulating the exporter elevates intracellular zinc and accelerates terminal differentiation, this further supports a paradigm in which a GATA/heme/zinc circuit is a vital determinant of erythrocyte development.

The term “inhibitory nucleic acid molecule” means a single stranded or double-stranded RNA or DNA, specifically RNA, such as triplex oligonucleotides, ribozymes, aptamers, small interfering RNA including siRNA (short interfering RNA) and shRNA (short hairpin RNA), antisense RNA, or a portion thereof, or an analog or mimetic thereof, that is capable of reducing or inhibiting the expression of a target gene or sequence. Inhibitory nucleic acids can act by, for example, mediating the degradation or inhibiting the translation of mRNAs which are complementary to the interfering RNA sequence. An inhibitory nucleic acid, when administered to a mammalian cell, results in a decrease (e.g., by 5%, 10%, 25%, 50%, 75%, or even 90-100%) in the expression (e.g., transcription or translation) of a target sequence. Typically, a nucleic acid inhibitor comprises or corresponds to at least a portion of a target nucleic acid molecule, or an ortholog thereof, or comprises at least a portion of the complementary strand of a target nucleic acid molecule. Inhibitory nucleic acids may have substantial or complete identity to the target gene or sequence, or may include a region of mismatch (i.e., a mismatch motif). The sequence of the inhibitory nucleic acid can correspond to the full-length target gene, or a subsequence thereof. In one aspect, the inhibitory nucleic acid molecules are chemically synthesized.

The specific sequence utilized in design of the inhibitory nucleic acids is a contiguous sequence of nucleotides contained within the expressed gene message of the target. Factors that govern a target site for the inhibitory nucleic acid sequence include the length of the nucleic acid, binding affinity, and accessibility of the target sequence. Sequences may be screened in vitro for potency of their inhibitory activity by measuring inhibition of target protein translation and target related phenotype, e.g., inhibition of cell proliferation in cells in culture. In general, it is known that most regions of the RNA (5′ and 3′ untranslated regions, AUG initiation, coding, splice junctions and introns) can be targeted using antisense oligonucleotides. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Phosphorothioate antisense oligonucleotides may be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. A peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Short interfering (si) RNA technology (also known as RNAi) generally involves degradation of an mRNA of a particular sequence induced by double-stranded RNA (dsRNA) that is homologous to that sequence, thereby “interfering” with expression of the corresponding gene. A selected gene may be repressed by introducing a dsRNA which corresponds to all or a substantial part of the mRNA for that gene. Without being held to theory, it is believed that when a long dsRNA is expressed, it is initially processed by a ribonuclease III into shorter dsRNA oligonucleotides of as few as 21 to 22 base pairs in length. Accordingly, siRNA may be affected by introduction or expression of relatively short homologous dsRNAs. Exemplary siRNAs have sense and antisense strands of about 21 nucleotides that form approximately 19 nucleotides of double stranded RNA with overhangs of two nucleotides at each 3′ end.

siRNA has proven to be an effective means of decreasing gene expression in a variety of cell types. siRNA typically decreases expression of a gene to lower levels than that achieved using antisense techniques, and frequently eliminates expression entirely. In mammalian cells, siRNAs are effective at concentrations that are several orders of magnitude below the concentrations typically used in antisense experiments.

The double stranded oligonucleotides used to effect RNAi are specifically less than 30 base pairs in length, for example, about 29, 28, 27, 26, 25, 24, 23, 22, 21, 20, 19, 18, or 17 base pairs or less in length, and contain a segment sufficiently complementary to the target mRNA to allow hybridization to the target mRNA. Optionally, the dsRNA oligonucleotide includes 3′ overhang ends. Exemplary 2-nucleotide 3′ overhangs are composed of ribonucleotide residues of any type and may be composed of 2′-deoxythymidine residues, which lowers the cost of RNA synthesis and may enhance nuclease resistance of siRNAs in the cell culture medium and within transfected cells. Exemplary dsRNAs are synthesized chemically or produced in vitro or in vivo using appropriate expression vectors. Longer RNAs may be transcribed from promoters, such as T7 RNA polymerase promoters, known in the art.

Longer dsRNAs of 50, 75, 100, or even 500 base pairs or more also may be utilized in certain embodiments. Exemplary concentrations of dsRNAs for effecting RNAi are about 0.05 nM, 0.1 nM, 0.5 nM, 1.0 nM, 1.5 nM, 25 nM, or 100 nM, although other concentrations may be utilized depending upon the nature of the cells treated, the gene target and other factors readily identifies by one of ordinary skill in the art.

Compared to siRNA, shRNA offers advantages in silencing longevity and delivery options. Vectors that produce shRNAs, which are processed intracellularly into short duplex RNAs having siRNA-like properties provide a renewable source of a gene-silencing reagent that can mediate persistent gene silencing after stable integration of the vector into the host-cell genome. Furthermore, the core silencing ‘hairpin’ cassette can be readily inserted into retroviral, lentiviral, or adenoviral vectors, facilitating delivery of shRNAs into a broad range of cell types.

A hairpin can be organized in either a left-handed hairpin (i.e., 5′-antisense-loop-sense-3′) or a right-handed hairpin (i.e., 5′-sense-loop-antisense-3′). The shRNA may also contain overhangs at either the 5′ or 3′ end of either the sense strand or the antisense strand, depending upon the organization of the hairpin. If there are any overhangs, they are specifically on the 3′ end of the hairpin and include 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Specifically, there is only one phosphate group on the 5′ end of the region that will form the antisense strand following Dicer processing. In one exemplary embodiment, a right-handed hairpin can include a 5′ end (i.e., the free 5′ end of the sense region) that does not have a 5′ phosphate group or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′-OH functional group (e.g., the 5′-most nucleotide is a 5′-deoxy nucleotide). In cases where the hairpin is a left-handed hairpin, preferably the 5′ carbon position of the 5′-most nucleotide is phosphorylated.

Hairpins that have stem lengths longer than 26 base pairs can be processed by Dicer such that some portions are not part of the resulting siRNA that facilitates mRNA degradation. Accordingly, the first region, which may include sense nucleotides, and the second region, which may include antisense nucleotides, may also contain a stretch of nucleotides that are complementary (or at least substantially complementary to each other), but are or are not the same as or complementary to the target mRNA. While the stem of the shRNA can include complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

Modified shRNAs can retain the modifications in the post-Dicer processed duplex. In exemplary embodiments, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin. Also, Dicer processing of hairpins with this configuration can retain the 5′ end of the sense strand intact, thus preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality. Examples of exemplary modification patterns are described in detail in U.S. Patent Publication No. 20050223427 and International Patent Publication Nos. WO 2004/090105 and WO 2005/078094, the disclosures of each of which are incorporated by reference herein in their entirety.

shRNA may comprise sequences that were selected at random, or according to a rational design selection procedure. For example, rational design algorithms are described in International Patent Publication No. WO 2004/045543 and U.S. Patent Publication No. 20050255487, the disclosures of which are incorporated herein by reference in their entireties. Additionally, it may be desirable to select sequences in whole or in part based on average internal stability profiles (“AISPs”) or regional internal stability profiles (“RISPs”) that may facilitate access or processing by cellular machinery.

Ribozymes are enzymatic RNA molecules capable of catalyzing specific cleavage of mRNA, thus preventing translation. The mechanism of ribozyme action involves sequence specific hybridization of the ribozyme molecule to complementary target RNA, followed by an endonucleolytic cleavage event. The ribozyme molecules specifically include (1) one or more sequences complementary to a target mRNA, and (2) the well-known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site-specific recognition sequences can be used to destroy target mRNAs, hammerhead ribozymes may alternatively be used. Hammerhead ribozymes cleave mRNAs at locations dictated by flanking regions that form complementary base pairs with the target mRNA. Specifically, the target mRNA has the following sequence of two bases: 5′-UG-3′. The construction and production of hammerhead ribozymes is well known in the art and is described more fully in U.S. Pat. No. 5,633,133, the contents of which are incorporated herein by reference.

Gene targeting ribozymes may contain a hybridizing region complementary to two regions of a target mRNA, each of which is at least 5, 6, 7, 8, 9, 10, 11, 12, 13, 14, 15, 16, 17, 18, 19, or 20 contiguous nucleotides (but which need not both be the same length).

Hammerhead ribozyme sequences can be embedded in a stable RNA such as a transfer RNA (tRNA) to increase cleavage efficiency in vivo. In particular, RNA polymerase III-mediated expression of tRNA fusion ribozymes is well known in the art. There are typically a number of potential hammerhead ribozyme cleavage sites within a given target cDNA sequence. Specifically, the ribozyme is engineered so that the cleavage recognition site is located near the 5′ end of the target mRNA— to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Ribozymes also include RNA endoribonucleases (“Cech-type ribozymes”) such as the one which occurs naturally in Tetrahymena thermophile, described in International Patent Publication No. WO 88/04300. The Cech-type ribozymes have an eight base pair active site which hybridizes to a target RNA sequence where after cleavage of the target RNA takes place. In one embodiment, Cech-type ribozymes target eight base-pair active site sequences that are present in a target gene or nucleic acid sequence.

Ribozymes can be composed of modified oligonucleotides (e.g., for improved stability, targeting, etc.) and can be chemically synthesized or produced through an expression vector. Because ribozymes, unlike antisense molecules, are catalytic, a lower intracellular concentration is required for efficiency. Additionally, in certain embodiments, a ribozyme may be designed by first identifying a sequence portion sufficient to cause effective knockdown by RNAi. Portions of the same sequence may then be incorporated into a ribozyme.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. Nucleic acid molecules to be used in triple helix formation for the inhibition of transcription are specifically single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Inhibitory nucleic acids can be administered directly or delivered to cells by transformation or transfection via a vector, including viral vectors or plasmids, into which has been placed DNA encoding the inhibitory oligonucleotide with the appropriate regulatory sequences, including a promoter, to result in expression of the inhibitory oligonucleotide in the desired cell. Known methods include standard transient transfection, stable transfection and delivery using viruses ranging from retroviruses to adenoviruses. Delivery of nucleic acid inhibitors by replicating or replication-deficient vectors is contemplated. Expression can also be driven by either constitutive or inducible promoter systems. In other embodiments, expression may be under the control of tissue or development-specific promoters.

Vectors may be introduced by transfection using carrier compositions such as Lipofectamine 2000 (Life Technologies) or Oligofectamine™ (Life Technologies). Transfection efficiency may be checked using fluorescence microscopy for mammalian cell lines after co-transfection of hGFP-encoding pAD3.

The effectiveness of the inhibitory oligonucleotide may be assessed by any of a number of assays, including reverse transcriptase polymerase chain reaction or Northern blot analysis to determine the level of existing human sclerostin mRNA, or Western blot analysis using antibodies which recognize the human sclerostin protein, after sufficient time for turnover of the endogenous pool after new protein synthesis is repressed.

As used herein, “nucleic acid” means single-, double-, or multiple-stranded DNA, RNA and derivatives thereof. In certain embodiments, the nucleic acid is single stranded. Modifications may include those that provide other chemical groups that incorporate additional charge, polarizability, hydrogen bonding, electrostatic interaction, and functionality to the nucleic acid. Such modifications include, but are not limited to, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping moieties. A 2′deoxy nucleic acid linker is a divalent nucleic acid of any appropriate length and/or internucleotide linkage wherein the nucleotides are 2′deoxy nucleotides.

Certain nucleic acid compounds can exist in unsolvated forms as well as solvated forms, including hydrated forms. In general, the solvated forms are equivalent to unsolvated forms. Certain nucleic acid compounds may exist in multiple crystalline or amorphous forms. In general, all physical forms are equivalent for the uses contemplated by the methods provided herein.

Certain nucleic acid compounds may possess asymmetric carbon atoms (optical centers) or double bonds; the racemates, diastereomers, geometric isomers and individual isomers.

In general, complementary nucleic acid strands hybridize under stringent conditions. The term “stringent hybridization conditions” or “stringent conditions” refers to conditions under which a nucleic acid hybridizes to an inhibitory nucleic acid, for example, to form a stable complex (e.g., a duplex), but to a minimal number of other sequences. The stability of complex is a function of salt concentration and temperature (See, for example, Sambrook et al., Molecular Cloning: A Laboratory Manual 2d Ed. (Cold Spring Harbor Laboratory, (1989); incorporated herein by reference). Stringency levels used to hybridize a nucleic acid to an inhibitory nucleic acid can be readily varied by those of skill in the art. The phrase “low stringency hybridization conditions” refers to conditions equivalent to hybridization in 10% formamide, 5 times Denhart's solution, 6 times SSPE, 0.2% SDS at 42° C., followed by washing in 1 times SSPE, 0.2% SDS, at 50° C. Denhart's solution and SSPE are well known to those of skill in the art as are other suitable hybridization buffers. (See, e.g., Sambrook et al.). The term “moderately stringent hybridization conditions” refers to conditions equivalent to hybridization in 50% formamide, 5 times Denhart's solution, 5 times SSPE, 0.2% SDS at 42° C., followed by washing in 0.2 times SSPE, 0.2% SDS, at 60° C. The term “highly stringent hybridization conditions” refers to conditions equivalent to hybridization in 50% formamide, 5 times Denhart's solution, 5 times SSPE, 0.2% SDS at 42° C., followed by washing in 0.2 times SSPE, 0.2% SDS, at 65° C.

“Complementary,” as used herein, refers to the capacity for precise pairing of two nucleobases (e.g., A to T (or U), and G to C) regardless of where in the nucleic acid the two are located. For example, if a nucleobase at a certain position of nucleic acid is capable of hydrogen bonding with nucleobases at a certain position of an inhibitory nucleic acid, then the position of hydrogen bonding between the nucleic acid and the inhibitory nucleic acid is considered to be a complementary position. The nucleic acid and inhibitory nucleic acid are “substantially complementary” to each other when a sufficient number of complementary positions in each molecule are occupied by nucleobases that can hydrogen bond with each other. Thus, the term “substantially complementary” is used to indicate a sufficient degree of precise pairing over a sufficient number of nucleobases such that stable and specific binding occurs between the nucleic acid and an inhibitory nucleic acid. The phrase “substantially complementary” thus means that there may be one or more mismatches between the nucleic acid and the inhibitory nucleic acid when they are aligned, provided that stable and specific binding occurs. The term “mismatch” refers to a site at which nucleobases in the nucleic acid and nucleobases in the inhibitory nucleic acid with which it is aligned are not complementary. The nucleic acid and inhibitory nucleic acid are “perfectly complementary” to each other when the nucleic acid is fully complementary to the inhibitory nucleic acid across the entire length of the nucleic acid.

Generally, a nucleic acid is “antisense” to an inhibitory nucleic acid when, written in the 5′ to 3′ direction, it comprises the reverse complement of the corresponding region of the target nucleic acid. “Antisense compounds” are also often defined in the art to comprise the further limitation of, once hybridized to a target, being able to modulate levels, expression or function of the target compound.

As used herein, “sequence identity” or “identity” refers to the nucleobases in two sequences that are the same when aligned for maximum correspondence over a specified comparison window. As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “pharmaceutically acceptable salts” is meant to include salts of the active compounds which are prepared with relatively nontoxic acids or bases, depending on the particular substituents found on the compounds described herein. When nucleic acid compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When nucleic acid compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Certain nucleic acid compounds contain both basic and acidic functionalities that allow the compounds to be converted into either base or acid addition salts.

The neutral forms of the nucleic acid compounds may be regenerated by contacting the salt with a base or acid and isolating the parent compound in the conventional manner. The parent form of the compound differs from the various salt forms in certain physical properties, such as solubility in polar solvents.

As used herein, “pharmaceutical composition” means therapeutically effective amounts of the compound together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

For topical application to the skin, the drug may be made up into a cream, lotion or ointment. Cream or ointment formulations which may be used for the drug are conventional formulations well known in the art. Topical administration includes transdermal formulations such as patches.

For topical application to the eye, the inhibitor may be made up into a solution or suspension in a suitable sterile aqueous or non-aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of controlling red blood cell production, comprising
contacting red blood cell precursors with a composition comprising a zinc chelator, a composition comprising an inhibitor of a zinc importer protein, or a combination thereof; wherein the contacting inhibits survival of the red blood cell precursors, inhibits terminal differentiation of the red blood cell precursors to mature red blood cells, or a combination thereof.

2. The method of claim 1, wherein the red blood cell precursors are cultured hematopoietic precursor cells or cultured precursor pluripotent stem cells.

3. The method of claim 1, wherein the method comprises administering a zinc chelator.

4. The method of claim 3, wherein the zinc chelator comprises (N,N,N′,N′-Tetrakis(2-Pyrimidyl)ethylenediamine) (TPEN), 10-phenanthroline, ethylene glycol tetraacetic acid (EGTA), diethyldithiocarbamate (DEDTC), ethylenediamine-N,N′-diacetic acid (EDDA), diethylenetriamine pentaacetic acid (DTPA), pyridine disulfide ethylenediaminetetraacetic acid (PSDE), amino-iminodiacetic acid (IDA), 2-amino-4-fluorophenol N,N,O triacetic acid (AFTA), a zinc-binding peptide, or a combination thereof.

5. The method of claim 3, wherein the zinc chelator comprises (N,N,N′,N′-Tetrakis(2-Pyrimidyl)ethylenediamine) (TPEN).

6. The method of claim 1, wherein the inhibitor of the zinc exporter protein is an inhibitory RNA.

7. The method of claim 6, wherein the inhibitory RNA is a small interfering RNA comprising 19 to 29 nucleotides that are substantially complementary to 19 to 29 nucleotides of human SLC39A8.

8. The method of claim 1, comprising inhibiting terminal differentiation of the red blood cell precursors to mature red blood cells, wherein the method further comprises removing the undifferentiated precursors from red blood cells terminally differentiated in vitro to provide red blood cells completely matured for transplantation therapy.

9. A method of treating a patient suffering from a disease associated with defective erythropoiesis, comprising
administering an effective amount of a composition comprising a zinc chelator, a composition comprising an inhibitor of a zinc importer protein, or a combination thereof;
wherein the administering inhibits survival of red blood cell precursors, inhibits terminal differentiation of red blood cell precursors to mature red blood cells, or a combination thereof.

10. The method of claim 9, wherein the disease associated with defective erythropoiesis is myelodysplastic syndromes (MDS), sickle cell anemia, or an anemia that is unresponsive to an erythropoiesis stimulating agent; and the method comprises administering zinc or an inhibitor of a zinc exporter protein.

11. The method of claim 10, wherein the inhibitor of a zinc exporter protein is a therapeutic RNA.

12. The method of claim 10, further comprising co-administering a chemotherapeutic agent.

13. The method of claim 9, wherein the disease associated with defective erythropoiesis is leukemia; and wherein the method comprises administering a zinc chelator or an inhibitor of a zinc importer protein.

14. The method of claim 9, wherein the inhibitor of a zinc importer protein is a therapeutic small inhibitory RNA.

15. The method of claim 9, wherein the zinc chelator comprises (N,N,N′,N′-Tetrakis(2-Pyrimidyl)ethylenediamine) (TPEN), 10-phenanthroline, ethylene glycol tetraacetic acid (EGTA), diethyldithiocarbamate (DEDTC), ethylenediamine-N,N′-diacetic acid (EDDA), diethylenetriamine pentaacetic acid (DTPA), pyridine disulfide ethylenediaminetetraacetic acid (PSDE), amino-iminodiacetic acid (IDA), 2-amino-4-fluorophenol N,N,O triacetic acid (AFTA), a zinc-binding peptide, or a combination thereof.

16. The method of claim 10, wherein the zinc chelator comprises (N,N,N′,N′-Tetrakis(2-Pyrimidyl)ethylenediamine) (TPEN).

17. A method of treating a human subject in need of a red blood cell transplant, comprising
ex vivo culturing of hematopoietic precursor cells or pluripotent stem cells in the presence of a composition comprising zinc, a composition comprising an inhibitor of a zinc exporter protein, or a combination thereof; wherein contacting either promotes survival of hematopoietic precursor cells or precursors from pluripotent stem cells, or promotes terminal differentiation of the hematopoietic precursor cells or precursors from pluripotent stem cells to mature, differentiated red blood cells;
isolating mature, differentiated red blood cells from the ex vivo culture; and
transplanting the mature, differentiated red blood cells into the human subject.

18. The method of claim 17, wherein the inhibitor of the zinc exporter protein is a therapeutic RNA.

19. The method of claim 17, wherein the human subject is suffering from a disease associated with defective erythropoiesis.
